(19) United States
(12) Patent Application Publication
(10) Pub. No.: US 2011/0229580 A1
(43) Pub. Date: Sep. 22, 2011

(54) COMPOSITIONS AND METHODS FOR NANO-IN-MICRO PARTICLES

(75) Inventors: Rohit SRIVASTAVA, Mumbai (IN); Abhijeet Balwantrao Joshi, Thane (IN); R. Keerthi PRASAD, Gurgaon (IN)

(73) Assignee: Indian Institute of Technology Bombay, School of Biosciences and Bioengineering

(21) Appl. No.: 12/728,936

(22) Filed: Mar. 22, 2010

(51) Int. Cl. A61K 9/16; A61K 31/573; A61J 3/00

(52) U.S. Cl. 424/493; 514/180; 264/4

(57) ABSTRACT

Disclosed herein are compositions, methods and kits for a microsphere with one or more entrapped nanoparticles. The method of preparation comprises atomizing a suspension comprising a polysaccharide and one or more nanoparticles into a solution comprising a cross linking agent.

### BACKGROUND

The development of nano-in-micro particulate matrices has opened new avenues in basic, applied scientific research and industrial applications. The nano-in-micro particulate matrices are attractive for many applications, for example, the pigment, paper, rubber, plastic and biomedical industries owing to their size, properties and capacity for spatial and temporal delivery of bioactives. Such matrices may exhibit properties which are different from those of individual components enabling them to provide dual functionality. Several methods have been developed for the production of nano-in-micro particulate matrices including emulsion polymerization, intercalative polymerization, and hybrid latex polymerization. All of these methods use surfactants and organic solvents, which have potential implications for the toxicity of the final product.

The preparation of microspheres using methods of gelation may not result in uniform microspheres owing to diffusion based gelling from outside to inside or vice versa.

The emulsification technique uses a non aqueous phase, a surfactant and an aqueous phase for formation of the emulsion in which alginate forms the aqueous part. Gelling is carried out by addition of divalent salts in the aqueous phase and then treatment with either aqueous or organic acids to break the emulsion to form microspheres. When calcium chloride is added to the stabilized emulsion system, it leads to disruption of the equilibrium of the system which causes clumping of particles.

There is a need for simple and efficient methods for making nano-in-micro particles.

### SUMMARY

In one aspect, there is provided a method of preparing a microsphere with one or more entrapped nanoparticles, comprising atomizing a suspension comprising a polysaccharide and one or more nanoparticles into a solution comprising a cross linking agent, thereby preparing a microsphere with one or more entrapped nanoparticles. In some embodiments, the polysaccharide is an alginate. In some embodiments, the alginate solution comprises from about 0.01 w/v concentration to about 5% w/v concentration of alginate.

In some embodiments, the nanoparticle comprises material selected from the group consisting of inorganic material, polymeric material, and combination thereof. In some embodiments, the inorganic material comprises hydroxyl apatite, calcium phosphate, calcium carbonate, gold, iron, silica and magnetic material. In some embodiments, the magnetic material is iron oxide. In some embodiments, the polymeric material comprises gelatin, poly(lactic-co-glycolic acid), and peroxalate.

In some embodiments, the nanoparticle is prepared by methods selected from the group consisting of desolvation, emulsification, atomization, precipitation, and sedimentation.

In some embodiments, the nanoparticle is a coated nanoparticle with multiple layers. In some embodiments, the coated nanoparticle comprises layers of poly cations and poly anions. In some embodiments, the multiple layer coated nanoparticle is made from layer by layer assembly of polyelectrolytes on the nanoparticle.

In some embodiments, the suspension and/or the nanoparticle comprises a fluorescent agent. In some embodiments, the fluorescent agent is selected from fluoresceinisothiocyanato-dextran (FITC-dextran), ruthenium based dye, platinum porphyrin, or combination thereof. In some embodiments, the fluorescent agent is fluoresceinisothiocyanato-dextran (FITC-dextran). In some embodiments, the fluorescent agent is ruthenium based dye or porphyrin.

In some embodiments, the suspension and/or the nanoparticle comprises one or more therapeutic agents.

In some embodiments, the suspension and/or the nanoparticle comprises an agent selected from the group consisting of: enzyme, virus, cell, spore, drug, protein, dye, ink, fragrance, flavor, and magnetic particle.

In some embodiments, a ratio of the nanoparticle and the polysaccharide is in a range of about 1:10 to about 10:1. In some embodiments, a ratio of the nanoparticle and the polysaccharide is in a range of about 1:5 to about 5:1. In some embodiments, a ratio of the nanoparticle and the polysaccharide is in a range of about 1:4 to about 1:1.

In some embodiments, a size of the nanoparticle ranges from about 1 nm to about 2000 nm. In some embodiments, a size of the nanoparticle ranges from about 1 nm to about 500 nm.

In some embodiments, a size of the microsphere is in a range of about 1 to about 130 μm. In some embodiments, a size of the microsphere is in a range of about 5 to about 60 μm.

In some embodiments, the cross linking agent is a divalent and/or trivalent metal salt. In some embodiments, the metal salt has a metal cation selected from the group consisting of: barium, lead, copper, strontium, cadmium, calcium, zinc, nickel, aluminium, and mixture thereof.

In some embodiments, the methods of the present technology further comprise adding a chemical reagent to the microsphere. In some embodiments, the chemical reagent removes the entrapped nanoparticles and gels the microsphere internally.

In some embodiments, the cross linking agent is calcium chloride. In some embodiments, a concentration of the cross linking agent in the solution is in a range of about 0.5% (w/v) to around 10% (w/v).

In some embodiments, the atomization comprises using a spray nozzle system of a droplet generator. In some embodiments, the atomization comprises syringe extrusion, coaxial air flow method, mechanical disturbance method, electrostatic force method, or electrostatic bead generator method. In some embodiments, the atomization comprises spraying the suspension through a nozzle of an air driven droplet generating encapsulation unit.

In some embodiments, a shape or a size of the microsphere is varied by varying one or more parameters selected from the group consisting of: nozzle diameter; flow rate of the spray; pressure of the spray; distance of the solution comprising the cross linking agent from the nozzle; concentration of the polysaccharide solution; and concentration of the cross linking agent.

In some embodiments, the suspension is sprayed with the flow rate of about 10 ml/min.

In some embodiments, the suspension is sprayed at an air pressure ranging from about 0 mbar-500 mbar. In some embodiments, the suspension is sprayed at an air pressure ranging from about 300 mbar-500 mbar.

In some embodiments, the microsphere is of spherical shape.

In some embodiments, size of the microsphere is in a range of about 5 to about 130 μm.

In one aspect, there is provided a composition comprising a microsphere as prepared by the methods of the present technology and an excipient. In some embodiments, there is provided a composition comprising a microsphere with one or more entrapped nanoparticles, where the microsphere is prepared by the method comprising atomizing a suspension comprising a polysaccharide and one or more nanoparticles into a solution comprising a cross linking agent; and an excipient.

In one aspect, there is provided an in vivo method for imaging a human or an animal subject comprising administering to the subject a diagnostically effective amount of a composition comprising a microsphere with one or more entrapped nanoparticles, wherein the microsphere with one or more entrapped nanoparticles is prepared by atomizing a suspension of a polysaccharide and one or more nanoparticles into a solution comprising a cross linking agent to yield the entrapped one or more nanoparticles, wherein the nanoparticle comprises a metal useful for imaging selected from the group consisting of iron, gadolinium, manganese, cobalt, copper, nickel, rhenium, technetium, and indium; and examining a body of the subject with a diagnostic device and compiling images of the body or parts thereof. In some embodiments, the diagnostic device is x-ray scanner, magnetic resonance imaging, or computerized axial tomography (CAT scan).

In one aspect, there is provided a method for treating cancer in a subject, comprising administering to a cancerous tissue in the subject a therapeutically effective amount of a composition comprising a microsphere with one or more entrapped nanoparticles, wherein the microsphere with the one or more entrapped nanoparticles is prepared by atomizing a suspension of a polysaccharide and one or more nanoparticles into a solution comprising a cross linking agent to yield the one or more entrapped nanoparticles, wherein the one or more entrapped nanoparticles comprise a magnetic material selected from the group consisting of: iron, iron oxide, copper, silver, gold, magnesium, molybdenum, lithium, tantalum, or combination thereof; and applying an alternating magnetic field to the cancerous tissue in the subject to generate heat to substantially kill the cancerous tissue.

In one aspect, there is provided a method for delivering one or more therapeutic drugs to a subject in need thereof, comprising administering to a subject a composition comprising a microsphere with one or more entrapped nanoparticles, wherein the microsphere with one or more entrapped nanoparticles is prepared by atomizing a suspension of a polysaccharide and one or more nanoparticles into a solution comprising a cross linking agent to yield the entrapped one or more nanoparticles, wherein the nanoparticle comprises one or more therapeutic drugs, thereby delivering the one or more therapeutic drugs to the subject.

In one aspect, there is provided an in vitro method for diagnosing an analyte in a sample, comprising administering to a sample a diagnostically effective amount of a composition comprising a microsphere with one or more entrapped nanoparticles, wherein the microsphere with one or more entrapped nanoparticles is prepared by atomizing a suspension of a polysaccharide and one or more nanoparticles into a solution comprising a cross linking agent to yield the entrapped one or more nanoparticles, wherein the nanoparticle comprises a fluorescent agent selected from the group consisting of fluoresceinisothiocyanato-dextran (FITC-dextran), ruthenium based dye, platinum porphyrin, or combination thereof, wherein the microsphere comprises an enzyme which enzyme upon reaction with a substrates in the sample activates the fluorescent agent; and examining the sample with a diagnostic device for sensing a fluorescence of the fluorescent agent. In some embodiments, the sample is blood, plasma, tissue, urine, feces, sweat, nasal discharge, mucus, or saliva. In some embodiments, the diagnostic device is a fluorescence detector. In some embodiments, the method diagnoses a level of glucose, lipid, or protein in the sample.

In one aspect, there is provided an in vitro or an in vivo method for tissue engineering, comprising contacting a tissue with a therapeutically effective amount of a composition comprising a microsphere with one or more entrapped nanoparticles, wherein the microsphere with one or more entrapped nanoparticles is prepared by atomizing a suspension of a polysaccharide and one or more nanoparticles into a solution comprising a cross linking agent to yield the entrapped one or more nanoparticles, wherein the nanoparticle comprises a material selected from the group consisting of calcium phosphate, hydroxyl apatite, and calcium carbonate; wherein the nanoparticle supports the tissue growth.

In one aspect, there is provided a method for delivering one or more macromolecules to a subject in need thereof, comprising administering to a subject a composition comprising a microsphere with one or more entrapped nanoparticles, wherein the microsphere with one or more entrapped nanoparticles is prepared by atomizing a suspension of a polysaccharide and one or more nanoparticles into a solution comprising a cross linking agent to yield the entrapped one or more nanoparticles, wherein the nanoparticle comprises one or more macromolecules. In some embodiments, the macromolecule is a protein or a gene.

In one aspect, there is provided a kit, comprising a microsphere as prepared in the methods of the present technology. In some embodiments, the kit further comprises instructions for use.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 depicts an illustrative schematic diagram of instrumental set up used for preparation of nano-in micro particles using an air driven droplet generator.

FIG. 2 depicts an illustrative embodiment of—I. Optical images of (a) alginate microspheres and (b) nano-in-micro system; II. Scanning Electron Microscopy (SEM) images of (a) uncoated gelatin nanoparticles, (b) coated gelatin nanoparticles, (c) uncoated calcium carbonate (CaCO₃) nanoparticles, (d) coated CaCO₃ nanoparticles and (e) typical nanoparticles-in-alginate microspheres; and III. TEM images of (a, c) uncoated and (b, d) coated nanoparticles of gelatin (a, b) and CaCO₃ (c, d).

FIG. 3 depicts an illustrative embodiment of a zeta potential measurement of gelatin and CaCO₃ nanoparticles during layer-by-layer (LBL) assembly (represented by horizontal bars) and variation of zeta potential of gelatin nanoparticles due to changes in pH (-▴-) (represented as line graph); X and Y error bars represent the standard error of mean values.

FIG. 4 depicts an illustrative embodiment of a Confocal Laser Scanning Microscopy (CLSM) images of (1a) fluorescien iso-thiocyanate conjugated dextran (FITC-dex) loaded gelatin nanoparticles, (1b) gelatin-in-alginate microspheres and (2a) CaCO₃ nanoparticles, and (2b) CaCO₃-in-alginate microspheres. Differential interference contrast images of (1c) gelatin-in-alginate microspheres and (2c) CaCO₃-in-alginate microspheres.

FIG. 5 depicts an illustrative embodiment of a Fourier Transform Infrared Spectroscopy (FTIR) spectra of gelatin nanoparticles (A); gelatin-in-alginate hybrid microspheres (B); alginate microspheres (C); CaCO₃-in-alginate hybrid microsphere (D); and CaCO₃ nanoparticles (E).

FIG. 6 depicts an illustrative embodiment of drug release studies for uncoated nanoparticles (-♦-), coated nanoparticles [1 BL (-▪-), 2 BL (-▴-)], alginate microspheres (--), nano-in-micro [1:4(-♦-), 3:4(-ll-)]. Y error bars represents the standard deviation for triplicate measurement.

FIG. 7 depicts an illustrative embodiment of an optical microscopic images of CaCO₃ nanoparticles at 60×.

FIG. 8 depicts an illustrative embodiment of Gaussian particle size distribution of CaCO₃ nanoparticles (a) intensity weighted and (b) volume weighted distributions.

FIG. 9 depicts an illustrative embodiment of SEM images of CaCO₃ nanoparticles, (a) bare nanoparticles; (b) polystyrene sulfonate (PSS) doped nanoparticles and (c) coated nanoparticles (2 BL).

FIG. 10 depicts an illustrative embodiment of TEM image of (a) uncoated PSS doped CaCO₃ nanoparticles and (b) LBL coated PSS doped CaCO₃ nanoparticles containing two bilayers of PSS and polyallylamine hydrochloride (PAH).

FIG. 11 depicts an illustrative embodiment of Zeta potential measurements of CaCO₃ nanoparticles during different stages of LBL self-assembly (y error bars represent the standard deviation for n=3 measurements, 1 BL: One bilayers and 2 BL: Two bilayers).

FIG. 12 depicts an illustrative embodiment of differential interference contrast (DIC) images of FITC-dextran encapsulated CaCO₃ nanoparticles for (I) 70 KDa, (II) 150 KDa and (III) 500 KDa and fluorescent microscopic images of FITC-dextran encapsulated CaCO₃ nanoparticles (b) unwashed (c) one washing (d) double washing for (I) 70 KDa, (II) 150 KDa and (III) 500 KDa (all images captured at 60×, image IIId captured at 60× with a zoom of 2.35 times).

FIG. 13 depicts an illustrative embodiment of FITC-dextran (70 KDa) release from uncoated, 1 BL coated and 2 BL coated CaCO₃ nanoparticles over a 8 day period (y error bars represent the standard deviation for n=3 measurements).

FIG. 14 depicts an illustrative embodiment of a comparison of X-ray diffraction (XRD) profiles of CaCO₃ nanoparticles formed at different concentration of PSS 0.5% (A), 0.25% (B), and 0% (C).

FIG. 15 depicts an illustrative embodiment of FT-IR spectra of (A) CaCO₃ nanoparticles, (B) PSS nanoparticles and (C) PSS doped CaCO₃ nanoparticles.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology is described herein using several definitions, as set forth throughout the specification. As used herein, unless otherwise stated, the singular forms “a,” “an,” and “the” include plural reference. Thus, for example, a reference to “a microsphere” includes a plurality of microspheres, and a reference to “a nanoparticle” is a reference to one or more nanoparticles.

A “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this technology or process steps to produce a composition or achieve an intended result. Embodiments defined by each of these transition terms are within the scope of this technology.

In one aspect, there is provided a method of preparing a microsphere with one or more entrapped nanoparticles, including atomizing a polysaccharide and nanoparticle suspension into a solution including a cross linking agent, thereby entrapping one or more nanoparticles in the microsphere. In one aspect, there are provided compositions containing microspheres with one or more entrapped nanoparticles, as prepared herein. Such entrapped or encapsulated or embedded or confined nanoparticle in the microsphere is also referred to herein as “nano-in-micro.” The nature and the concentration of the cross linking agent is as described herein. In some embodiments of the present technology, the microspheres with one or more entrapped nanoparticles are prepared without using a surfactant. In some embodiments of the present technology, the microspheres with one or more entrapped nanoparticles are surfactant free. Examples of such surfactants include, but are not limited to, sodium dodecyl sulfate and phospholipids. In alternative embodiments, gelatin and PVA are used alone or in combination with polysaccharide in the method of preparing a microsphere with one or more entrapped nanoparticles as provided herein. Not wishing to be bound by theory, nanoparticles embedded in microspheres can also be considered as a hybrid matrix because they can contain two different types of materials in a single matrix. Further, nanoparticles embedded in microspheres can also be considered as a composite material(s) where materials are intimate mixtures of different components. These views are not mutually exclusive as composite material may comprise a hybrid matrix, or vice versa. The present technology is useful to produce nanoparticles embedded in microsphere, where, depending on the composition, they may be viewed as comprising hybrid matrix and/or composite microsphere.

An “entrapped” refers to embedded or confined or encapsulated (partially or completely) inside something, for example a microsphere or a nanoparticle. A “suspension” refers to a fluid containing solid particles which may be same particles or different particles.

In some embodiments, the atomization process of the present technology provides microspheres having controllable shape and/or size. The different sized microspheres can be produced by changing instrumental and sample parameters and may depend on a scale of batch. Several instrumental parameters that can be varied include, but are not limited to, air pressure, nozzle size, flow rate, distance of cross linking solution form the nozzle, and sample parameters that can be varied include, but are not limited to, concentration of polysaccharide, concentration of nanoparticles, concentration of the cross linking agent, and ratio of mixing.

The method of the present technology to prepare a microsphere with one or more entrapped nanoparticles, is more industrially feasible in comparison to a commonly used emulsification method. Atomization method provides several advantages for scaling to industrial level including, but not limited to, continuous processing (in comparison to batch process of emulsification), reduced instrumentation requirements with changes in size requirements for different types of microspheres, and reductions in the number of steps of production (such as, without limitation, formation of emulsion, breaking emulsion, separation of organic phase, separation of micro-spheres), thereby reducing time and cost.

Encapsulation of the nanoparticle in the microsphere provides various benefits over microspheres or nanoparticles individually. Nanoparticles by themselves may not be stable being prone to degradation and may attract immunological responses. Microspheres can be immobilized in a particular region and are less prone to degradation by the reticulo endothelial system (RES). The microspheres with one or more entrapped nanoparticles of the present technology have benefits of both nanoparticulate systems and nanoparticles. In some embodiments, the microspheres with one or more entrapped nanoparticles provide a delivery system wherein the nanoparticle provides an increased surface area for the encapsulation of a drug or any other agent and/or the microsphere system provides protection from the reticulo-endothelial system and an increase in retention time.

In some embodiments, the microsphere with one or more entrapped nanoparticles is prepared by providing a fine spray of a nanoparticle/polysaccharide suspension through a nozzle of an encapsulation unit, into a well-stirred cross linking solution, which cross links the polysaccharide droplets to form the microsphere coating.

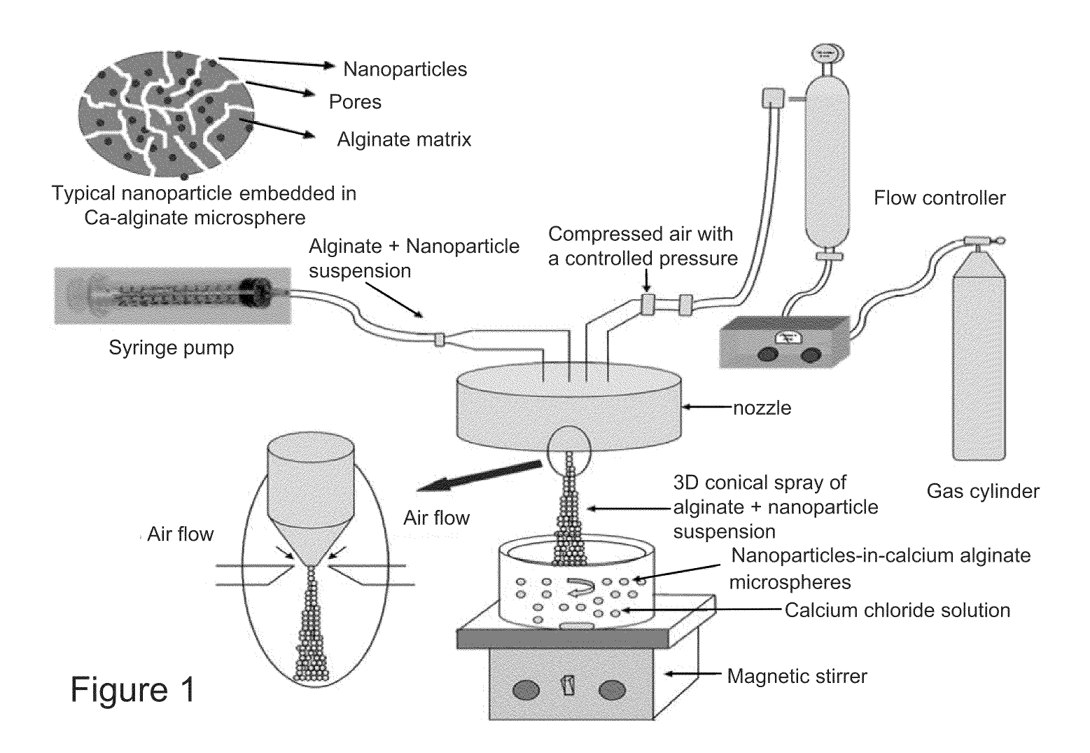

FIG. 1 depicts an illustrative embodiment of the methods of the present technology for preparing the microsphere entrapped with one or more nanoparticles. FIG. 1 is a schematic diagram of instrument used for preparation of nano-in micro particles using an air driven droplet generator. In some embodiments, the method comprises blending of polysaccharide into a liquid medium which forms the basis of the polymeric material. Such liquid medium includes, but is not limited to, water, gelatin cross linked by glutaraldehyde, and polyvinyl alcohol (PVA) cross linked by borax. The polysaccharide solution may optionally be mixed with a suspension of insoluble salt of metal cations using magnetic stirrer, until uniform dispersion is formed. In order to prepare the microsphere with entrapped nanoparticle, the desired nanoparticle suspension is added to the polymeric solution. The suspension of the polysaccharide and the nanoparticle is then transferred to a syringe in the syringe pump. The suspension is then sprayed using an encapsulation unit through a spray nozzle based on coaxial air flow system.

Some of the parameters of the atomization of the methods of the present technology that can be varied to obtain different sizes of microspheres include, but are not limited to, flow rate, height of the nozzle head and air pressure, as illustrated in Table 1.

TABLE 1
Illustrative instrumental parameters

| | Parameters | | | | | |
|---|---|---|---|---|---|---|
| | Alginate | Flow | | CaCl₂ | | Mean |
| S. | conc. | rate | Pressure | conc. | Distance | Particle |
| No. | (% w/v) | (ml/hr) | (mbar) | (mM) | (cm) | size (μm) |
| 1 | 2.0 | 20 | 300 | 200 | 10 | 22 |
| 2 | 2.0 | 20 | 200 | 200 | 10 | 43 |
| 3 | 2.0 | 20 | 100 | 200 | 10 | 60 |
| 4 | 2.0 | 15 | 300 | 200 | 10 | 18 |
| 5 | 2.0 | 15 | 200 | 200 | 10 | 37 |
| 6 | 2.0 | 15 | 100 | 200 | 10 | 55 |
| 7 | 2.0 | 10 | 300 | 200 | 10 | 12 |
| 8 | 2.0 | 10 | 200 | 200 | 10 | 28 |
| 9 | 2.0 | 10 | 100 | 200 | 10 | 38 |
| 10 | 2.0 | 10 | 500 | 200 | 10 | 8 |

As illustrated in Table 1, in some embodiments, the instrument parameters may be varied to achieve the desired shape and/or size of the microsphere of the present technology. In some embodiments, as the pressure at which the suspension is sprayed is reduced while keeping the flow rate constant, the size of the microsphere increases. In some embodiments, when the flow rate of the suspension is reduced while keeping the pressure constant, the size of the microsphere decreases. In some embodiments, by varying the flow rate and/or the pressure of the spray, the sphericity of the microsphere can be controlled.

In some embodiments, the sample parameters may be varied to achieve the desired shape and/or size of the microsphere. For example, when nanoparticles are mixed in the polysaccharide solution, smaller particle sizes could be formed. Without being limited by any theory, it is believed to be mainly due to production of shear during atomization. In some embodiments, the nanoparticle may provide a nuclei for formation of droplets which in turn may lead to formation of smaller particles.

In some embodiments, the atomization comprises using a spray nozzle system of a droplet generator. The suspension is sprayed using encapsulation unit into calcium chloride (CaCl₂) solution with a desired flow rate and air pressure. The microspheres of the present technology may be prepared using a droplet generator, for which the parameters like flow rate of the polysaccharide solution such as sodium alginate solution, concentration of sodium alginate, concentration of cross slinking agent such as calcium chloride, distance of the nozzle from the surface of liquid, air pressure, etc. are varied in order to get a desired size range. For example, in some embodiments, the suspension is sprayed with a flow rate of about 5 ml/hour to about 20 ml/hour. In some embodiments, the suspension is sprayed with a flow rate of about 5 ml/hour to about 10 ml/hour; or alternatively about 10 ml/hour to about 1 ml/hour; or alternatively about 10 ml/hour to about 20 ml/hour; or alternatively about 10 ml/hour to about 15 ml/hour.

In some embodiments, the suspension is sprayed with an air pressure of ranging from about 0 mbar-500 mbar. In some embodiments, the suspension is sprayed with an air pressure ranging from about 0 mbar-400 mbar; or alternatively about 0 mbar-300 mbar; or alternatively about 0 mbar-200 mbar; or alternatively about 0 mbar-100 mbar; or alternatively about 100 mbar-500 mbar; or alternatively about 100 mbar-400 mbar; or alternatively about 100 mbar-300 mbar; or alternatively about 100 mbar-200 mbar; or alternatively about 200 mbar-500 mbar; or alternatively about 200 mbar-400 mbar; or alternatively about 200 mbar-300 mbar; or alternatively about 300 mbar-500 mbar; or alternatively about 300 mbar-400 mbar; or alternatively about 400 mbar-500 mbar.

In some embodiments, atomization may include the use of methods such as, but not limited to, syringe extrusion, coaxial air flow method, mechanical disturbance method, electrostatic force method, or electrostatic bead generator method.

Atomization of a suspension of polysaccharide material and preformed nanoparticles using a spray nozzle system of a droplet generator into a well stirred cross linking solution leads to formation of cross linked microspheres. In air driven atomization, liquid droplets may be broken into fine droplets with the aid of air flow pressure. The air flow pattern can be altered to form coaxial pattern to form uniform nanoparticles. Coaxial air flow technique uses concentric streams of air which shear the liquid droplets released from one or more needles. The size of particles generated may be controlled by factors such as, air flow velocity, viscosity of encapsulant and the distance of the needle to the solution of the cross linking agent.

Alternatives to the air driven mechanism are electrostatic field, mechanical disturbance and electrostatic force. Electrostatic mechanism utilizes a potential difference between a capillary tip such as a nozzle and a flat counter electrode to reduce the diameter of the droplets by applying an additional force (i.e. electric force) in the direction of gravitational force in order to overcome the upward capillary force of liquid. These can be used to produce small droplets <100 μm from highly viscous liquids depending on their conductivity. In mechanical disturbance method, liquid droplets are broken into fine droplets using a mechanical disturbance. Typically, vibrations are used for producing the mechanical disturbance. In electrostatic force method, electrostatic forces destabilize a viscous jet, where the electrostatic force is used to disrupt the liquid surface instead of a mechanical disturbance.

After sufficient time for cross linking, the microspheres are collected using centrifugation or filtration and are washed until free from the chemical reagents. The composite nanoparticles are then centrifuged and subjected to duplicate or triplicate washing cycles.

In some embodiments, the presence of inorganic material or the cross linking agent in the polysaccharide suspension may increase the shear experienced by the droplets leading to formation of smaller microspheres. The presence of the cross linking agent inside the polysaccharide microsphere causes the internal gelation when the hardened microspheres are treated chemically with acidic reagents or chelating agents to remove the entrapped inorganic component. The action of acid liberates free metal cations which gels the polysaccharide (alginate) microspheres internally. The chemical reagents used to dissolve the entrapped inorganic component include, but are not limited to, a chelating agent like ethylene diaminetetraacetic acid (EDTA), an aqueous acid like hydrochloric acid (HCl), sulfuric acid (H₂SO₄) or an organic acid like acetic acid.

The concentration of the polysaccharide in the solution may be dependent on the viscosity of the solution and the nozzle aperture used for spraying the solution. Without being limited by any theory, an increase in the viscosity of the polysaccharide solution may decrease the flow-ability of the suspension, and may result in blockade of the nozzle. In some embodiments, the blockade of the nozzle may be prevented by reducing the viscosity of the suspension or by reducing the concentration of the polysaccharide in the solution. In some embodiments, the viscosity of the suspension in the present technology is in such a way that no deformation of the microspheres takes place after atomization.

In some embodiments, the polysaccharide is an alginate solution. In some embodiments, gelatin or polyvinyl alcohol (PVA) is used to prepare gelatin microsphere or PVA microsphere with one or more entrapped nanoparticles. In some embodiments, gelatin or PVA is used instead of the polysaccharide in the suspension, in the methods of the present technology.

In some embodiments, the polysaccharide solution or the gelatin solution or the PVA solution contains about 0.01 (w/v) concentration to about 5% (w/v) concentration of the polysaccharide or gelatin or PVA, respectively. In some embodiments, the polysaccharide solution or the gelatin solution or the PVA solution contains about 0.01-1% w/v; or alternatively about 0.01-2% w/v; or alternatively about 0.01-3% w/v; or alternatively about 0.01-4% w/v; or alternatively 0.5-1% w/v; or alternatively about 0.5-2% w/v; or alternatively about 0.5-3% w/v; or alternatively about 0.5-4% w/v; or alternatively about 1-2% w/v; or alternatively about 1-3% w/v; or alternatively about 1-4% w/v; or alternatively about 1-5% w/v; or alternatively about 2-3% w/v; or alternatively about 2-4% w/v; or alternatively about 2-5% w/v; or alternatively about 3-4% w/v; or alternatively about 3-5% w/v; or alternatively about 4-5% w/v, concentration of alginate or gelatin or PVA, respectively.

In some embodiments, the cross linking agent that polymerizes or gels the sprayed polysaccharide solution internally, can be a solution of divalent/trivalent metal salt. The gelling agents such as metal cations can be used for cross linking of alginates. The cross linking agents of the present technology include, but are not limited to, metal cations of barium, lead, copper, strontium, cadmium, calcium, zinc, nickel and aluminium or a mixture of any of the foregoing. The cross linking ability may be influenced by the type of the salt chosen for gelling. In some embodiments, the selection of the cross linking agent may depend on the molecular size of the cations and the corresponding counter ions. Carbonate, sulphate, chloride, acetate, etc. are some of the counter ions for the above listed cations. In some embodiments, the cations with higher molecular size may give a less cross linked polysaccharide in comparison to the cations with low molecular size. In some embodiments, the selection of the cross linking agent may depend on the toxicity of the cations. For example, in an in vivo application, the toxicity of the cation may need to be evaluated. An example of the cation that may be toxic in in vivo applications includes, aluminium cation. In some embodiments of the present technology, the cross linking agent is calcium chloride.

In some embodiments, a concentration of the cross linking agent in the solution is about 0.5% w/v to about 10% w/v. In some embodiments, the concentration of the cross linking agent in the solution is about 0.5-8% w/v; or alternatively about 0.5-5% w/v; or alternatively about 0.5-3% w/v; or alternatively about 0.5-2% w/v; or alternatively about 0.5-1% w/v; or alternatively about 1-10% w/v; or alternatively about 1-8% w/v; or alternatively about 1-5% w/v; or alternatively about 1-2% w/v; or alternatively about 1-3% w/v; or alternatively about 1-4% w/v; or alternatively about 2-3% w/v; or alternatively about 2-4% w/v; or alternatively about 2-5% w/v; or alternatively about 2-8% w/v; or alternatively about 2-10% w/v; or alternatively about 3-4% w/v; or alternatively about 3-5% w/v; or alternatively about 3-8% w/v; or alternatively about 3-10% w/v; or alternatively about 4-5% w/v; or alternatively about 5-10% w/v; or alternatively about 5-6% w/v; or alternatively about 6-8% w/v; or alternatively about 8-10% w/v.

In some embodiments, the nanoparticle of the present technology comprises at least one material including, but not limited to, inorganic material, polymeric material, and combination thereof. In some embodiments, the inorganic material includes, without limitation, hydroxyl apatite, calcium phosphate, calcium carbonate, gold, iron, silica and magnetic material. In some embodiments, the magnetic material includes, but is not limited to, diamagnetic material, paramagnetic material, and ferromagnetic material. Illustrative embodiments of such magnetic materials include, but are not limited to, iron (Fe), iron oxide (Fe₃O₄), copper, silver, gold, magnesium, molybdenum, lithium, tantalum and combination thereof. In some embodiments, the polymeric material includes, but is not limited to, gelatin, poly(lactic-co-glycolic acid), and peroxalate.

In some embodiments, the nanoparticles of the present technology are made using a layer by layer assembly (LBL) method. In some embodiments, the nanoparticle is a coated nanoparticle with multiple layers. In some embodiments, the coated nanoparticle comprises layers of poly cations and poly anions. In some embodiments, the multiple layer coated nanoparticle is made from layer by layer assembly of polyelectrolytes on the nanoparticle.

In some embodiments, the layer-by-layer (LBL) assembly method is used to achieve desired release profile of the agent from the entrapped nanoparticles in the microsphere. In some embodiments, during the release of the active agent from inside the nanoparticles, the entrapped nanoparticles remain inside the microsphere. In other embodiments, during the release of the active agent from inside the nanoparticles, the entrapped nanoparticles are released from inside the microsphere. A series of cationic or anionic substances of a polyelectrolyte are used for assembling multilayers on the nanoparticles. These polyelectrolytes are used at appropriate concentration prepared in an inorganic material including, but not limited to, calcium chloride. Depending on the surface charge of the nanoparticles, the nanoparticles are dispersed in oppositely charged polyelectrolyte solution for predefined time, followed by at least one or two consecutive centrifugation and washing steps to remove excess polyelectrolyte. The polyelectrolyte coated nanoparticles are suspended in appropriate polyelectrolyte solutions. The reaction is allowed for pre-defined time prior to centrifugation and washing steps. The process is repeated to form layer by layer assembly. An illustrative embodiment of the LBL technology is as described in the example herein.

In some embodiments, the nanoparticles of the present technology are prepared by a desolvation method. Other methods for the preparation of nanoparticles include, but are not limited to, emulsification, atomization, sedimentation, dispersion and precipitation methods. In emulsification, the aqueous solution is mixed in a non-aqueous phase containing an emulsifier to form emulsion droplets. The solution is then gelled with a gelling agent. In the dispersion method, direct dispersion of polymeric solution in a cross linking solution leads to formation of nanoparticles. This method can be used for the preparation of chitosan nanoparticles where in the chitosan is dissolved in acetic acid and the solution is poured in the polyphosphate solution. In the sedimentation/precipitation method, mixing of two counter-ions leads to formation of nanoparticles.

Various examples of the methods of preparation of nanoparticles are as described below. Gelatin nanoparticles can be made through desolvation, salting out or emulsification methods as known in literature, preferably through desolvation method. Gelatin nanoparticles can be prepared by a two-step desolvation method. A gelatin solution is prepared at room temperature and is desolvated by slowly adding an equal volume of acetone, a non-solvent for gelatin, and is kept for sedimentation. The supernatant is discarded and the sediment is re-dissolved in water adjusted to pH 2.5. The nanoparticles are formed during the second desolvation step where acetone in added drop-wise under constant stirring. Afterwards, gluteraldehyde may be added to harden the formed particles. The solution is kept under constant stirring for specified time. Purification is done by a three-fold centrifugation and re-suspension in acetone: water mixture and the final pellet is suspended in milliQ water and stored at 4-8° C.

Calcium carbonate (CaCO₃) nanoparticles can be formed by precipitation reaction of calcium chloride (CaCl₂) and sodium carbonate (Na₂CO₃). The formed CaCO₃ nanoparticles are separated and washed by centrifugation using milliQ water.

Peroxalate nanoparticles are formed from a reaction between the 4-hydroxy benzyl alcohol, 1,8-octane diol, oxalyl chloride. The polymer is dissolved in dichloromethane and poured in poly vinyl alcohol. The organic solvent is evaporated in a rotavapor and polymeric nanoparticles are centrifuged. The suspension is washed using milli Q water to remove excess surfactant.

PLGA nanoparticles are prepared using emulsification method by dissolving PLGA in acetone. The resulting solution is then poured in surfactant solution such as, Tween 20. The excess acetone is evaporated in a rotavapor. The nanoparticles are washed using centrifugation.

In some embodiments of the methods of the present technology, a ratio of the nanoparticle and the polysaccharide in the suspension is in a range of about 1:10 to about 10:1. In some embodiments, the ratio of the nanoparticle and the polysaccharide in the suspension is in a range of about 1:9 to about 9:1; or alternatively in the range of about 1:8 to about 8:1; or alternatively in the range of about 1:7 to about 7:1; or alternatively in the range of about 1:6 to about 6:1; or alternatively in the range of about 1:5 to about 5:1; or alternatively in the range of about 1:4 to about 4:1; or alternatively in the range of about 1:3 to about 3:1; or alternatively in the range of about 1:2 to about 2:1; or alternatively in the range of about 1:4 to about 3:4; or alternatively about 1:1, 1:2, 1:3, 1:4, 1:5, 1:6, 1:8, 2:1, 3:1, 4:1, 5:1, 2:3, 3:2, 3:4, 5:4, 7:3, 9:5, etc.

In some embodiments of the methods of the present technology, a size of the nanoparticle is in a range of about 1 nm to about 2000 nm. In some embodiments of the methods of the present technology, a size of the nanoparticle is in a range of about 1 nm to about 1500 nm; or alternatively about 1 nm to about 1000 nm; or alternatively about 1 nm to about 900 nm; or alternatively about 1 nm to about 800 nm; or alternatively about 1 nm to about 700 nm; or alternatively about 1 nm to about 600 nm; or alternatively about 1 nm to about 500 nm; or alternatively about 1 nm to about 400 nm; or alternatively about 1 nm to about 300 nm; or alternatively about 1 nm to about 200 nm; or alternatively about 1 nm to about 100 nm. In some embodiments of the methods of the present technology, a size of the nanoparticle is 1 nm, 5 nm, 50 nm, 100 nm, 200 nm, or greater than 1 nm.

In some embodiments of the methods of the present technology, a size of the microsphere is in a range of about 1 μm to about 130 μm. In some embodiments, the size of the microsphere is in a range of about 1 μm to about 100 μm; or alternatively about 1 μm to about 80 μm; or alternatively about 1 μm to about 50 μm; or alternatively about 1 μm to about 25 μm; or alternatively about 1 μm to about 10 μm; or alternatively about 1 μm to about 5 μm; or alternatively about 5 μm to about 130 μm; or alternatively about 5 μm to about 100 μm; or alternatively about 5 μm to about 50 μm; or alternatively about 50 μm to about 100 μm; or alternatively about 75 μm to about 100 μm; or alternatively about 5 μm, 10 μm, 20 μm, 40 μm, 50 μm, 100 μm, or 130 μm.

The encapsulated nanoparticles embedded or entrapped in a polysaccharide based microsphere can contain either a drug or any other agent or a combination of a drugs or agents, as described herein, encapsulated/adsorbed/confined within them. Alternatively, the polysaccharide and nanoparticle suspension may include a drug or an agent that is also encapsulated in the microsphere during the encapsulation process. Alternatively, both the microsphere as well as the nanoparticle encapsulated inside the microsphere may include a drug or an agent, as described herein.

In some embodiments of the methods of the present technology, the suspension and/or the nanoparticle comprises a fluorescent agent. Fluorescent agents are well known in the art. Examples of fluorescent agent include, but are not limited to, fluoresceinisothiocyanato-dextran (FITC-dextran), ruthenium based dye, or platinum porphyrin or a mixture thereof.

In some embodiments of the methods of the present technology, the suspension and/or the nanoparticle comprises one or more therapeutic agents. Examples of therapeutic agents include, but are not limited to, anti-cancer agents, such as, but not limited to, taxanes, alkylating agents, anthracyclines, epothilones, topoisomerase II inhibitors, etc.; analgesics such as, but are not limited to, ibuprofen, acetoaminophen, etc.; anesthetics; hormone or a steroid; anti-microbial such as leptomycin or erythromycin, etc.; anti-diarrheal agents, such as, but are not limited to, opioids, loperamide, octreotide, etc.; immunosuppressive agents, such as, but are not limited to, natalizumab, mitoxantrone, azathioprine, etc.; anti-inflammatory agents, such as, but not limited to, dexametasome; and the like.

In some embodiments of the methods of the present technology, the suspension and/or the nanoparticle comprises one or more agents including, but are not limited to, enzyme, virus, cell, spore, drug, protein, dye, ink, fragrance, flavor, and magnetic particles. The matrices for custom applications, such as enzymes, cells, spores, drugs, proteins, dyes, inks, fragrances, flavors, etc. can be encapsulated in the composite microspheres depending on the stabilizing environment required for active ingredients.

In some embodiments of the methods of the present technology, the suspension and/or the nanoparticle comprises one or more macromolecules. Examples of macromolecule include, but are not limited to, proteins, enzymes, gene, and the like.

In some embodiments the suspension for preparing microspheres as described above, further comprise a macromolecule or an agent, as described herein, loaded on to the cross linking agent, prior to mixing with polysaccharide solution.

### Method of Use

In another aspect of the present technology, the microspheres with one or more entrapped nanoparticles prepared as described herein, are used in various applications. Some of these applications are as described below. A “subject” of diagnosis or treatment is a mammal, including a human. Non-human animal subjects for diagnosis or treatment include, but are not limited to, murine, such as rats, mice, canine, such as dogs, leporids, such as rabbits, livestock, sport animals, and pets.

In some embodiments, there is provided an in vivo method for imaging a human or an animal subject or cells of any organism by administering a diagnostically effective amount of a composition comprising a microsphere with one or more entrapped nanoparticles. The microsphere with one or more entrapped nanoparticles is prepared by methods described herein. The nanoparticle in the imaging method comprises one or more metals useful for imaging including, but not limited to, gold, iron, gadolinium, manganese, cobalt, copper, nickel, rhenium, technetium, and indium. After the administration of the microsphere with one or more entrapped nanoparticles, the body of the subject is examined with a diagnostic device and images of the body or parts thereof are compiled. These images are analyzed using diagnostic devices including, but not limited to, X-ray scanner, magnetic resonance imaging (MRI), and/or computerized axial tomography (CAT scan).

A “diagnostically effective amount” refers to the amount of a microsphere or composition of the present technology to facilitate a desired diagnostic result. Diagnostics includes testing that is related to the in vitro, ex vivo, or in vivo diagnosis of disease states or biological status (e.g. diabetic, glucose intolerance, iron deficiency etc.) in mammals, for example, but not limited to, humans. The effective amount will vary depending upon the specific microsphere or composition used, the dosing regimen, timing of administration, the subject and disease condition being diagnosed, the weight and age of the subject, the severity of the disease condition, the manner of administration and the like, all of which can be determined readily by one of ordinary skill in the art.

These imaging methods, as described herein, can be used to diagnose or monitor treatment for conditions such as, but are not limited to, brain tumor; tumors of the chest, abdomen or pelvis; heart problems such as blockage; diseases of the liver, such as cirrhosis; diagnosis of other abdominal organs, including the bile ducts, gallbladder, and pancreatic ducts; cysts and solid tumors in the kidneys and other parts of the urinary tract; blockages or enlargements of blood vessels, including the aorta, renal arteries, and arteries in the legs; tumors and other abnormalities of the reproductive organs (e.g., uterus, ovaries, testicles, prostate); causes of pelvic pain in women, such as fibroids, endometriosis and adenomyosis; suspected uterine congenital abnormality in women undergoing evaluation for infertility; breast cancer; and breast implants.

In some embodiments, the metal useful for imaging in the foregoing embodiment is gadolinium, technetium or rhenium.

In some embodiments, there is provided a method for treating cancer in a subject by administering to a cancerous tissue in the subject a therapeutically effective amount of a composition comprising a microsphere with one or more entrapped nanoparticles. The microsphere with the one or more entrapped nanoparticles is prepared by methods described herein. The one or more entrapped nanoparticles comprise a magnetic material such as, but is not limited to, iron (Fe), iron oxide (Fe₃O₄), copper, silver, gold, magnesium, molybdenum, lithium, tantalum, or combination thereof. An alternating magnetic field is then applied to the cancerous tissue in the subject to generate heat to partially or substantially damage, decrease the growth, decrease the viability, induce apoptosis, and/or kill the cancerous tissue.

A “therapeutically effective amount” refers to the amount of a microsphere or composition of the present technology to induce a desired biological and/or therapeutic result. That result can be alleviation or modification of the signs, symptoms, or causes of a disease, or any other desired alteration of a biological system. The effective amount will vary depending upon the specific microsphere or composition used, the dosing regimen, timing of administration, the subject and disease condition being treated, the weight and age of the subject, the severity of the disease condition, the manner of administration and the like, all of which can be determined readily by one of ordinary skill in the art.

A “treating,” “treatment” and the like refer to obtaining a desired pharmacologic and/or physiologic effect. The effect can be prophylactic in terms of completely or partially preventing a disease or disorder or sign or symptom thereof, and/or can be therapeutic in terms of a partial or complete cure for a disorder and/or adverse effect attributable to the disorder. Examples of “treatment” include but are not limited to: preventing a disease from occurring in a subject that may be predisposed or at risk of a disease, but has not yet been diagnosed as having it; inhibiting a disease, i.e., arresting its development; and/or relieving or ameliorating the symptoms of disease or reducing the likelihood of recurrence of the disease. As is understood by those skilled in the art, “treatment” can include systemic amelioration of the symptoms associated with the pathology and/or a delay in onset of symptoms.

Iron oxide is an example of a magnetic material in the nanoparticle. Other magnetic materials are known in the art and are well within the scope of the present technology. Magnetic nanoparticles respond thermally to an alternating magnetic field and this local thermal response can be used in cancer treatments. Magnetic particles embedded in microspheres can be locally administered to the cancer tissue and subjected to alternating magnetic field to generate heat, which kills the cancer cells.

The example of cancers include, but are not limited to, solid tumors including malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer, non-small cell carcinoma of the lung, and cancer of the small intestine. The cancer can be a carcinoma, a sarcoma, a myeloma, a leukemia, a lymphoma or a mixed type.

Exemplary cancers include, but are not limited to: Digestive/gastrointestinal cancers such as anal cancer; bile duct cancer; extrahepatic bile duct cancer; appendix cancer; carcinoid tumor, gastrointestinal cancer; colon cancer; colorectal cancer, childhood; esophageal cancer; esophageal cancer, childhood; gallbladder cancer; gastric (stomach) cancer; gastric (stomach) cancer, childhood; hepatocellular (liver) cancer, adult (primary); hepatocellular (liver) cancer, childhood (primary); extrahepatic; pancreatic cancer; pancreatic cancer, childhood; sarcoma, rhabdomyosarcoma; pancreatic cancer, islet cell; rectal cancer; and small intestine cancer; endocrine cancers such as islet cell carcinoma (endocrine pancreas); adrenocortical carcinoma; adrenocortical carcinoma, childhood; gastrointestinal carcinoid tumor; parathyroid cancer; pheochromocytoma; pituitary tumor; thyroid cancer; thyroid cancer, childhood; multiple endocrine neoplasia syndrome, childhood; and carcinoid tumor, childhood; eye cancers such as intraocular melanoma; and retinoblastoma; musculoskeletal cancers such as Ewing's family of tumors; osteosarcoma/malignant fibrous histiocytoma of the bone; rhabdomyosarcoma, childhood; soft tissue sarcoma, adult; soft tissue sarcoma, childhood; clear cell sarcoma of tendon sheaths; and uterine sarcoma; breast cancer such as breast cancer and pregnancy; breast cancer, childhood; and breast cancer, male; neurologic cancers such as brain stem glioma, childhood; brain tumor, adult; brain stem glioma, childhood; cerebellar astrocytoma, childhood; cerebral astrocytoma/malignant glioma, childhood; ependymoma, childhood; medulloblastoma, childhood; pineal and supratentorial primitive neuroectodermal tumors, childhood; visual pathway and hypothalamic glioma, childhood; other childhood brain cancers; adrenocortical carcinoma; central nervous system lymphoma, primary; cerebellar astrocytoma, childhood; neuroblastoma; craniopharyngioma; spinal cord tumors; central nervous system atypical teratoid/rhabdoid tumor; central nervous system embryonal tumors; and supratentorial primitive neuroectodermal tumors, childhood and pituitary tumor; genitourinary cancers such as bladder cancer; bladder cancer, childhood; kidney cancer; ovarian cancer, childhood; ovarian epithelial cancer; ovarian low malignant potential tumor; penile cancer; prostate cancer; renal cell cancer, childhood; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; germ cell cancers such as extracranial germ cell tumor, childhood; extragonadal germ cell tumor; ovarian germ cell tumor; and testicular cancer; head and neck cancers such as lip and oral cavity cancer; oral cancer, childhood; hypopharyngeal cancer; laryngeal cancer; laryngeal cancer, childhood; metastatic squamous neck cancer with occult primary; mouth cancer; nasal cavity and paranasal sinus cancer; nasopharyngeal cancer; nasopharyngeal cancer, childhood; oropharyngeal cancer; parathyroid cancer; pharyngeal cancer; salivary gland cancer; salivary gland cancer, childhood; throat cancer; and thyroid cancer; hematologic/blood cell cancers such as a leukemia (e.g., acute lymphoblastic leukemia, adult; acute lymphoblastic leukemia, childhood; acute myeloid leukemia, adult; acute myeloid leukemia, childhood; chronic lymphocytic leukemia; chronic myelogenous leukemia; and hairy cell leukemia); a lymphoma (e.g., AIDS-related lymphoma; cutaneous T-cell lymphoma; Hodgkin's lymphoma, adult; Hodgkin's lymphoma, childhood; Hodgkin's lymphoma during pregnancy; non-Hodgkin's lymphoma, adult; non-Hodgkin's lymphoma, childhood; non-Hodgkin's lymphoma during pregnancy; mycosis fungoides; sezary syndrome; T-cell lymphoma, cutaneous; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders); lung cancer such as non-small cell lung cancer; and small cell lung cancer; respiratory cancers such as malignant mesothelioma, adult; malignant mesothelioma, childhood; malignant thymoma; thymoma, childhood; thymic carcinoma; bronchial adenomas/carcinoids; pleuropulmonary blastoma; non-small cell lung cancer; and small cell lung cancer; skin cancers such as Kaposi's sarcoma; Merkel cell carcinoma; melanoma; and skin cancer, childhood; other childhood cancers and cancers of unknown primary site; and metastases of the aforementioned cancers.

In some embodiments, there is provided a method for delivering one or more therapeutic drugs to a subject in need thereof by administering to the subject a composition comprising a microsphere with one or more entrapped nanoparticles. The microsphere with one or more entrapped nanoparticles is prepared as described in methods herein. The nanoparticle in the foregoing embodiment comprises one or more therapeutic drugs. Some examples of therapeutic agents are as described above. It is to be understood that the microspheres of the present technology can be used to deliver any therapeutic agent known in the art and such therapeutic agents are well within the scope of the present technology.

The microspheres of the present technology are applicable in developing sensors for diagnosis. In some embodiments, there is provided an in vitro method for diagnosing an analyte or a substrate in a sample, by administering to the sample (or providing the sample to the nano-in-micro sensor system) a diagnostically effective amount of a composition comprising a microsphere with one or more entrapped nanoparticles. The microsphere with one or more entrapped nanoparticles is prepared as described in methods herein. The nanoparticle in the foregoing embodiment may include a fluorescent agent (or other appropriate detection agent) including, but not limited to, fluoresceinisothiocyanate-dextran (FITC-dextran), ruthenium based dye, platinum porphyrin, or combination thereof. The microsphere in the foregoing embodiment may include an enzyme configured to activate the fluorescent agent as a result of a reaction with a substrate or the analyte (the substance that is being detected as part of the diagnostic test) in the sample. The sample is then examined with a device for detecting a fluorescence of the fluorescent agent. The presence of the fluorescence may indicate the presence or absence of the analyte in the sample, depending on the nature of the analyte.

The microspheres with one or more entrapped nanoparticles of the present technology, provide two or more compartments which are part of a diagnosing or a sensor mechanism. In some embodiments, the nanoparticle may contain fluorescent agent while the microsphere matrix may contain the enzyme which on reaction with substrate of interest activates the fluorescent agent and act as a sensor. In some embodiments, the microsphere may contain fluorescent agent while the nanoparticle may contain the enzyme which on reaction with substrate of interest activates the fluorescent agent and act as a sensor. In some embodiments, the demarcation between the positioning of the substances between the two compartments may not be clear and/or the substances may be present in both compartments. A two compartment system is also suitable for a combination of sensor and drug/protein/gene delivery, as described below.

The CaCO₃ micro/nanoparticle-enzyme encapsulated systems of the present technology can serve as matrix for wide applications including the development of enzyme based biosensors. The nano-in-micro particles of the present technology can provide an inert environment for maintenance of bio-functionality and activity of enzymes. High loading efficiency and stability inside the nano-in-micro particles can promote their usage in biosensor development with capability of detection of analytes in a selective and specific manner. Further, they can be used for instant analysis of biochemicals under real time conditions using invasive or minimally invasive techniques. The nano-in-micro particles of the present technology provide advantages including, but are not limited to, low cost, rapid and simple analytical tool, biocompatibility and biodegradability.

In some embodiments, the sample in the foregoing embodiments is blood, plasma, tissue, urine, feces, sweat, nasal discharge, mucus, saliva or interstitial fluid or any fluid from body. In some embodiments, the diagnostic device is a fluorescence detector.

In some embodiments, there is provided an in vitro, ex vivo, or an in vivo method for tissue engineering by contacting a tissue with a therapeutically effective amount of a composition comprising a microsphere with one or more entrapped nanoparticles. The microsphere with one or more entrapped nanoparticles is prepared as described herein. The nanoparticle in the foregoing embodiment comprises a material including, but is not limited to, e.g., calcium phosphate, hydroxyl apatite, and calcium carbonate. The nano-in-micro particle in the foregoing embodiment can also include growth promoting materials such as, but are not limited to, growth factors. The materials support the tissue growth, assists in tissue regeneration, and aids the mechanical properties of the composites.

In some embodiments, there is provided a method for delivering one or more macromolecules to a subject in need thereof, by administering to the subject a composition comprising a microsphere with one or more entrapped nanoparticles. The microsphere with one or more entrapped nanoparticles is prepared by methods described herein. The nanoparticle in the foregoing embodiment comprises one or more macromolecules.

The examples of macromolecules include, but are not limited to, enzymes, proteins, genes, etc. Various macromolecules can be loaded onto nanoparticles which are in turn embedded in microsphere. Inherently unstable macromolecules can be stabilized in an environment of nanoparticles and provide more effective targeted delivery.

### Pharmaceutical Formulations and Routes of Administration

In one aspect of the present technology, there is provided a composition comprising the microsphere with entrapped one or more nanoparticles of the present technology. The compositions of the present technology can be delivered directly or in pharmaceutical compositions along with suitable carriers or excipients, as is well known in the art. The methods of treatment of the present technology comprise administration of an effective amount of the microsphere of the technology to a subject in need. In a preferred embodiment, the subject is a mammalian subject, and in a most preferred embodiment, the subject is a human subject.

An effective amount of such microspheres can readily be determined by routine experimentation, including the effective and convenient route of administration, and the appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the therapeutic agent, dictate the type of formulation and the route of administration to be used, as well as whether local or systemic delivery would be preferred.

Pharmaceutical dosage forms of the nano-in-micro formulation of the present technology may be provided in an instant release, controlled release, sustained release, or target drug-delivery system. Commonly used dosage forms include, for example, solutions and suspensions, (micro-) emulsions, ointments, gels and patches, tablets, dragees, soft or hard shell capsules, suppositories, ovules, implants, amorphous or crystalline powders, aerosols, and lyophilized formulations. Depending on route of administration used, special devices may be required for application or administration of the microspheres, such as, for example, syringes and needles, inhalers, pumps, injection pens, applicators, or special flasks.

One or multiple excipients, also referred to as inactive ingredients, can be added to the microspheres to improve or facilitate manufacturing, stability, administration, and safety of the microspheres, and can provide a means to achieve a desired drug release profile. Therefore, the type of excipient(s) to be added to the microspheres can depend on various factors, such as, for example, the physical and chemical properties of the microspheres, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

Pharmaceutical dosage forms of the microspheres of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the microspheres can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The microspheres may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the nano-in-micro formulation of the present technology can be administered topically, such as through a skin patch, a semi-solid, or a liquid formulation, for example a gel, a (micro-) emulsion, an ointment, a solution, a (nano/micro)-suspension, or a foam. The penetration of the microspheres into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of the microspheres. Transdermal or topical administration would be preferred, for example, in situations in which local delivery with minimal systemic exposure is desired.

For administration by inhalation, or administration to the nose, the microspheres of the present technology are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the microspheres and a suitable powder base such as lactose or starch.

Compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of the microspheres, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the microspheres are well-known in the art and include water-based solutions containing a base, such as, for example, sodium hydroxide, to form an ionized compound; sucrose or sodium chloride as a tonicity agent; and a buffer, for example, a buffer that contains phosphate. Co-solvents, such as, for example, polyethylene glycols, may be added. These water-based systems are effective at dissolving the microspheres of the present technology and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of the microspheres, e.g. the nano-in-micro of the present technology containing a therapeutic agent, refers to that amount of the microsphere that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the microsphere or pharmaceutical composition that will elicit the biological or medical response of a tissue, system, animal or human that is being sought by the researcher, veterinarian, medical doctor or other clinician. Dosages particularly fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each microspheres but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of the microspheres administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the microspheres. Such a pack or device may, for example, comprise metal or plastic foil, such as a blister pack; or glass and rubber stoppers such as in vials. The pack or dispenser device may be accompanied by instructions for administration. Compositions comprising the microspheres of the present technology formulated in a compatible pharmaceutical carrier may also be prepared, placed in an appropriate container, and labeled for treatment of an indicated condition.

### Kits

In one aspect of the present technology, there is provided a kit, comprising a microsphere with one or more entrapped nanoparticles, as prepared by the methods of the present technology.

The kits may further comprise suitable packaging and/or instructions for use of the microsphere. Kits may also comprise a means for the delivery of the microsphere, such as tablets, syringe, catheter, or other such devices. The kits may further comprise surgical tools.

The kits may also include other compounds for use in conjunction with the microsphere described herein. Such compounds include alcohol, analgesics, anesthetics, antiseptics, etc. These compounds can be provided in a separate form. The kits may include appropriate instructions for the delivery of the microsphere with one or more entrapped nanoparticles, side effects, and any other relevant information. The instructions can be in any suitable format, including, but not limited to, printed matter, videotape, or computer readable disk.

In one embodiment, there is provided a kit comprising a microsphere with one or more entrapped nanoparticles, as prepared by the methods of the present technology; packaging; and instructions for use.

Unless otherwise stated all temperatures are in degrees Celsius. Also, in these examples and elsewhere, abbreviations have the following meanings:

| | |
|---|---|
| cm = | centimeter |
| g = | gram |
| HCl = | hydrochloric acid |
| hr = | hour |
| KV = | kilovolt |
| M = | molar |
| mg = | milligram |
| min. = | minute |
| μl = | microliter |
| μM = | millimolar |
| μm = | micrometer |
| ml = | milliliter |
| mV = | millivolt |
| NaCl = | sodium chloride |
| NaOH = | sodium hydroxide |
| nm = | nanometer |
| w/v = | weight/volume |

The following examples are provided to illustrate select embodiments of the technology as disclosed and claimed herein.

### EXAMPLES

The present technology is further illustrated by the following examples, which should not be construed as limiting in any way.

### Example 1

### Preparation of Gelatin Nanoparticles Embedded in Alginate Microspheres

In this study, gelatin nanoparticles embedded in microspheres (nano-in-micro) were developed using atomization. Dexamethasone loaded gelatin nanoparticles, and alginate based nano-in micro system were prepared and characterized using optical microscopy, SEM, TEM, DLS, Zeta Potential, CLSM and FTIR. Layer-by-Layer (LBL) assembly was used to control the release of entrapped materials from the systems. The drug release and macromolecular release from the nano-in-micro system was compared against uncoated and LBL coated nanoparticles. The results of the study show that spherical, non-aggregating, nano-in-micro particles (5-60 μm) can be prepared using atomization technique. The nano-in-micro systems can be used as drug release vehicles, biosensors and multifunctional systems.

Experimental

Materials

Gelatin [300 bloom (type A, from porcine skin) having mol. wt 87.5 kDa], alginate (low viscosity, 2%), sodium poly (styrene sulfonate) (PSS, 70 kDa), poly (allylamine hydrochloride) (PAH, 70 kDa), glutaraldehyde (25% solution), dexamethasone phosphate disodium salt (mol. wt. 392.5), sodium azide, and phosphate buffer saline tablets, were purchased from Sigma aldrich (India). Dialysis membrane (molecular cut off 10-14 kDa) was purchased from Hi Media Laboratories, India. Analytical reagents like acetone, HCl, NaOH and NaCl were purchased from SD Fine Chemicals, India. MilliQ water having resistance less than 18 mΩ was used in all process of preparation and washing of particles. All chemicals were analytical reagent grade and were used as received.

### Preparation of Gelatin Nanoparticles

Gelatin nanoparticles were prepared from a modified protocol of two-step desolvation method as developed by Azarmi et al. (Azarmi, S. (2006). Optimization of a two-step desolvation method for preparing gelatin nanoparticles and cell uptake studies in 143B osteosarcoma cancer cells. Journal of Pharmacy and Pharmaceutical Science, 9 (1), 124-132). Briefly, 25 ml of 5% gelatin solution was prepared at ambient temperature. Addition of equal volume of acetone to gelatin solution leads to formation of desolvated gelatin which could be sedimented. The desolvated gelatin was re-dissolved in water and pH adjusted to 2.5.

A second desolvation step was also carried out in order to prepare gelatin nanoparticles, the process including the addition of 75 ml acetone drop wise under constant stirring at 500 rpm. 250 μl of glutaraldehyde was added after 10 min to form cross linked gelatin nanoparticles. Parameters like temperature, stirring speed, precipitation time and speed of acetone addition were varied for preparation of nanoparticles. Nanoparticle preparation employed the use of some parameters including temperature (40° C.), stirring speed (500-700 r. p. m), precipitation time (90 sec) and speed of acetone addition (3-5 ml/min). The formed nanoparticles were purified thrice by centrifugation (30000 g for 15 min) and re-suspension in acetone:water (70:30) mixture. The washed and purified nanoparticles were suspended in milliQ water and stored at 4-8° C.

### Preparation of Drug Loaded Gelatin Nanoparticles

Dexamethasone sodium phosphate (0.2-0.5 mg/ml) was dissolved in water and mixed with de-solvated gelatin polymer after the first desolvation and precipitation. Second desolvation step was performed after drug loading as described in the previous section.

### Preparation of Nano-in-Micro System

The nano-in-micro system was prepared by using a commercial air driven droplet generator as shown in FIG. 1. The process involved mixing of nanoparticle suspension into a solution of 2% w/v sodium alginate. The nanoparticle-alginate suspension was then sprayed through an encapsulation unit or droplet generator (Nisco encapsulation unit Var J30, Zurich, Switzerland) defining several useful instrumental parameters (Nozzle diameter, flow rate, pressure, and distance of cross linker solution from nozzle) and sample parameters (concentration of alginate, concentration of CaCl₂, ratio of nanoparticles, alginate, concentration of nanoparticles). The flow rate and pressure were monitored and fixed according to the in-built program of the syringe pump (Multi-Phaser™, model NE-1000, New Era Pump Systems, NY). Several steps were carried out to fix the instrumental parameters like Nozzle diameter (0.35μ) flow rate of solution/suspension (18-20 ml/hr), pressure was maintained at (70-75 mbar) and distance of nozzle to cross linking solution (CaCl₂) (10 cm). The fine spray of alginate solution/nanoparticle suspension was collected into 250 mM CaCl₂ solution for gelation under constant stirring (250 rpm) for 20 minutes. The loaded microspheres obtained were separated by centrifugation and washed using double distilled water.

### Layer by Layer (LBL) Self-Assembly on Gelatin Nanoparticles

Solutions of polyethylene imine (PEI) (cationic) and PSS (anionic) were used for assembling [PSS/PEI]₂ multilayers on gelatin nanoparticles. These polyelectrolytes were used at 2 mg/ml concentration prepared in 250 mM calcium chloride. Depending on the surface charge of the nanoparticles, they were first dispersed in oppositely charged polyelectrolyte in 2 ml of either PEI or PSS solution for 20 min, followed by two consecutive centrifugation and washing steps to remove excess polyelectrolyte. PSS-coated gelatin nanoparticles were then suspended in PSS and PEI solutions, respectively. The reaction was allowed for 20 min prior to centrifugation and washing steps. The process was repeated to form gelatin [PSS-PEI]₂ assembly.

### Example 2

### Preparation of CaCO₃ Nanoparticles Embedded in Alginate Microspheres

In this study, CaCO₃ nanoparticles embedded in microspheres (nano-in-micro) were developed using atomization. FITC-dextran loaded CaCO₃ nanoparticles, and alginate based nano-in micro system were prepared and characterized using optical microscopy, SEM, TEM, DLS, Zeta Potential, CLSM and FTIR. Layer-by-Layer (LBL) assembly was used to control the release of entrapped materials from the systems. The macromolecular release from the nano-in-micro system was compared against uncoated and LBL coated nanoparticles. The results of the study show that spherical, non-aggregating, nano-in-micro particles (5-60 μm) can be prepared using atomization technique. The nano-in-micro systems are useful as drug release vehicles, biosensors and multifunctional systems.

### Experimental

### Materials

Alginate (low viscosity, 2%), sodium poly (styrene sulfonate) (PSS, 70 kDa), poly (allylamine hydrochloride) (PAH, 70 kDa) and fluorescien iso thiocyanate (70 kDa) were purchased from Sigma aldrich (India). Dialysis membrane (molecular cut off 10-14 kDa) was purchased from Hi Media Laboratories, India. Analytical reagents like acetone, HCl, NaOH and NaCl were purchased from SD Fine Chemicals, India. Calcium chloride (CaCl₂) and Sodium Carbonate (Na2CO3) were purchased from Merck Ltd, Mumbai, India. MilliQ water having resistance less than 18 mΩ was used in all process of preparation and washing of particles. All chemicals were analytical reagent grade and were used as received.

### Preparation of CaCO₃ Nanoparticles

CaCO₃ nanoparticles were prepared using precipitation from supersaturated solutions in presence of polystyrene sulfonate (PSS) at ambient temperature as investigated by Joshi et al. (Joshi and Srivastava (2009). Polyelectrolyte Coated Calcium Carbonate Nanoparticles as Templates for Enzyme Encapsulation. Advanced Science Letters, 2, 1-8). Briefly, 1 M sodium carbonate (Na₂CO₃) solution was rapidly poured into an equal volume of 1 M solution of CaCl₂ in the presence of 0.25% PSS in distilled water at room temperature with intense stirring and aged for 5 min. The particles so formed were thoroughly washed with de-ionized water and separated by centrifugation at 5200 g for 10 min.

### Preparation of FITC-Dex Loaded CaCO₃ Nanoparticles

FITC-dex (70 kDa) (0.2 mg/ml) was mixed with preformed CaCO₃ nanoparticles and incubated for 10 minutes. The FITC-dex loaded CaCO₃ nanoparticles were purified by centrifugation at 5200 g for 10 min and then washing to remove unloaded FITC-dex.

### Preparation of Nano-in-Micro System

The nano-in-micro system was prepared by using a commercial air driven droplet generator as shown in FIG. 1. The process involved mixing of nanoparticle suspension into a solution of 2% w/v sodium alginate. The nanoparticle-alginate suspension was then sprayed through an encapsulation unit or droplet generator (Nisco encapsulation unit Var J30, Zurich, Switzerland) defining several useful instrumental parameters (Nozzle diameter, flow rate, pressure, and distance of cross linker solution from nozzle) and sample parameters (concentration of alginate, concentration of CaCl₂, ratio of nanoparticles, alginate, concentration of nanoparticles). The flow rate and pressure were monitored and fixed according to the in-built program of the syringe pump (Multi-Phaser™, model NE-1000, New Era Pump Systems, NY). Several steps were carried out to fix the instrumental parameters like Nozzle diameter (0.35μ) flow rate of solution/suspension (18-20 ml/hr), pressure was maintained at (70-75 mbar) and distance of nozzle to cross linking solution (CaCl₂) (10 cm). The fine spray of alginate solution/nanoparticle suspension was collected into 250 mM CaCl₂ solution for gelation under constant stirring (250 rpm) for 20 minutes. The loaded microspheres obtained were separated by centrifugation and washed using double distilled water.

### Layer by Layer (LBL) Self-Assembly on Nanoparticles

Solutions of poly allyl amine (PAH) (cationic) and PSS (anionic) were used for assembling [PSS/PAH]₂ multilayers on CaCO3 nanoparticles. These polyelectrolytes were used at 2 mg/ml concentration prepared in 250 mM calcium chloride. Depending on the surface charge of the nanoparticles they were first dispersed in oppositely charged polyelectrolyte in 2 ml of either PAH or PSS solution for 20 min, followed by two consecutive centrifugation and washing steps to remove excess polyelectrolyte. PSS-coated CaCO3 nanoparticles were then suspended in PAH solutions, respectively. The reaction was allowed for 20 min prior to centrifugation and washing steps. The process was repeated to form gelatin [PSS-PAH]₂ assembly.

### Example 3

### Characterization of Particles of Example 1 and 2

### A. Methods

### Particle Size Analysis

Samples including gelatin nanoparticles, PSS doped CaCO₃ nanoparticles, alginate microspheres and gelatin-alginate/CaCO₃-alginate (Nano-in-micro system) microspheres were examined at 10× using an optical microscope (Leica DMIL, USA) with a digital camera attachment. The particle size and morphology of the microsphere was studied using Leica image analysis software. SEM images of micro/nanoparticles were obtained by placing them on top of carbon tape, the samples were sputtered with gold using a gold sputter coater, and measurements were conducted at 150-3000× magnifications using accelerating voltage of 3 KV in a Scanning electron microscope (Hitachi S-3400, Japan). TEM imaging of coated samples was carried out for morphological characterization.

One-drop of freshly made coated micro/nanoparticles were added on carbon coated film copper grid. The samples were allowed to dry using an infra-red (IR) radiation drier for 1 hour. The grids were then placed in the TEM (PHILIPS, CM200, USA) and viewed using an applied voltage of 200 KV. A small volume of nanoparticles were suspended in 1-2 ml of milliQ water. Each sample measurement was reported as mean diameter analyzed in triplicate. Particle size distributions of nanoparticles were measured using DLS (Brookhaven Instruments, USA). The technique was based on the scattering of incident laser light due to the random Brownian motion of the nanoparticles which can be plotted as correlation function against time. The nanoparticles size distribution was calculated using mathematical contin algorithm depending on the environment of sampling and the temperature.

### Zeta Potential Analysis (Electrophoretic Mobility) Measurement

The electrophoretic mobility of the uncoated and LBL coated microspheres were measured using Zetaplus (Brookhaven Instruments, USA). The ζ-potential was calculated from the electrophoretic mobility (μ) using the Smoluchowski relation: ζ=μη/ε (where η and ε are the viscosity and permittivity of the solvent, respectively). For this experiment, 50 μl sample solution containing the microspheres was diluted in 2 ml of distilled water and used for analysis. The measurements were reported as average value of triplicate measurements.

### Confocal Laser Scanning Microscopy (CLSM)

Fluorescent images of FITC-dex (70 kDa) loaded CaCO₃ nanoparticles were examined using confocal microscopy. Confocal micrographs of micro/nanoparticles were obtained with a flow view confocal laser scanning microscope (CLSM) equipped with a krypton-argon laser (Olympus FluoView™, Japan). An inverted microscope was also used which was equipped with an oil immersion objective lens (40×). The standard filter settings for fluorescence excitation (488 nm) and emission (520 nm) were used.

### Fourier Transform Infra-Red Spectroscopy (FTIR)

Samples including gelatin nanoparticles, CaCO₃ nanoparticles, alginate microspheres, gelatin-in-alginate hybrid microspheres and CaCO₃-in-alginate hybrid microspheres were obtained in their dry form. Samples were thoroughly ground with dried KBr and discs were prepared by compression. FTIR analysis of the samples was performed using FTIR spectrometer (Nicolet Instruments Corporation, Magna 550, USA). Spectra were obtained on the spectrometer from 400 to 4000 cm⁻¹.

### Encapsulation Efficiency

The actual drug loading or encapsulation efficiency (% EE) (in percentage) in the gelatin nanoparticles was determined by calculating the difference between the total (Wₜₒₜₐₗ) and the free drug (W_{free}) concentrations in the nanoparticle suspension and the supernatant per mg of gelatin nanoparticle (M).

$$\% EE = \frac{{W}_{Total} - {W}_{free}}{M} * 100 \qquad Formula 1$$

### Drug Release Studies

In-vitro drug release studies were performed on uncoated nanoparticles, polyelectrolyte coated nanoparticles cross linked and nanoparticle-in-alginate microspheres using a dialysis membrane with molecular weight cut-off of 10-14 KDa. Drug loaded uncoated, coated and microspheres were transferred to a beaker containing 200 ml of 0.01M phosphate buffered saline (PBS, pH 7.4) and 0.01% w/v sodium azide. The samples (in duplicate) were incubated in a 37° C. incubator under sink condition for the release studies. At preset time intervals, the release medium was collected and replaced with a fresh buffer solution. The % cumulative release profiles were obtained by taking the ratio of the amount of drug released to the total drug content and was determined spectrophotometrically at λmax of 242 nm. All measurements were performed for n=3 samples.

Macromolecular Release from CaCO₃ Nanoparticles

Encapsulated nanoparticles were prepared by loading FITC-dextran (70 KDa, 150 KDa and 500 KDa) (0.2 mg/ml), separately onto preformed nanoparticles and aged with stirring for 5 min. The FITC-dextran loaded particles were then separated by centrifugation at 5200 g for 10 min. FITC-dextran encapsulation in the particles was studied by an indirect method using fluorescence spectrophotometer (Hitachi F-2500, Japan) by analyzing the supernatant after loading and centrifugation. Encapsulation efficiency was determined using a previously prepared calibration curve for the fluorescence emission at different concentrations of FITC-dextran in the range of 0-0.2 mg/ml. FITC-dextran loaded CaCO₃ nanoparticles were also visualized using fluorescent imaging. Similarly, the enzyme loaded nanoparticles were centrifuged and supernatant was analyzed using fluorescence spectrophotometry.

FITC-dextran (70 KDa) loaded uncoated and coated nanoparticles were subjected to a time dependent release of FITC-dextran from the encapsulated matrix, which was monitored using fluorescence spectrophotometer, by ratiometric analysis of the supernatant and standard solution. Briefly, the supernatant was obtained by centrifugation of FITC-dextran loaded CaCO₃ nanoparticles at 5200 g for 10 min and fluorescence emission was acquired at an excitation wavelength of 488 nm and compared with standard solution of labeled macromolecule using fluorescence spectrophotometer. The ratio of fluorescence emission of supernatant and standard solution was compared against time for different batches of uncoated, one bilayer coated and two bilayer coated nanoparticles to estimate the FITC-dextran release.

### B. Results and Discussion

### Preparation of Nanoparticles

Desolvation process for preparing gelatin nanoparticles is a self-charge neutralization process where the positively charged segments in the chain overlap with the negatively charged segment of the same chain due to couloumbic interactions causing charge neutralization. Acetone and ethanol are commonly used desolvating agents. A two step desolvation process was selected because gelatin, unlike proteins such as albumin, is a mixture of protein fractions of different molecular weights and therefore different fractions precipitate at different degrees of desolvation. The first desolvation step involves elimination of low molecular weight fractions and the second step results in the actual formation of nanoparticles. The gelatin nanoparticles are formed largely through inter and intra molecular electrostatic interactions. The initial stages of nanoparticle formation occurs due to competition between intra-molecular folding and intermolecular aggregate formation.

CaCO₃ nanoparticles were prepared using a method of precipitation by a reaction of counter-ions and showed aggregation phenomena to a size of 5-7 μm. While not intending to be limited by any theory, the mechanism is believed to involve the formation of an amorphous precipitate which transforms into micro crystals of different morphologies and sizes. However when PSS was added to the reaction mixture, small spherical particles with uniform size of 500 nm to 2 μm were formed. Again, while not intending to be limited by any theory, he mechanism of reduction of size and formation of spherical nanoparticles can be explained on the basis of a micellization effect of PSS. The nanoparticles were selected due to their suitability of encapsulation for macromolecules due to their meso-porous structure (Joshi and Srivastava supra.; and Kawaguchi (1992). Crystallization of inorganic compounds in polymer solutions. I: Control of shape and form of calcium carbonate. Colloid and Polymer Science, 270 (12), 1176-1181).

### Preparation of Nano-in-Micro Particles

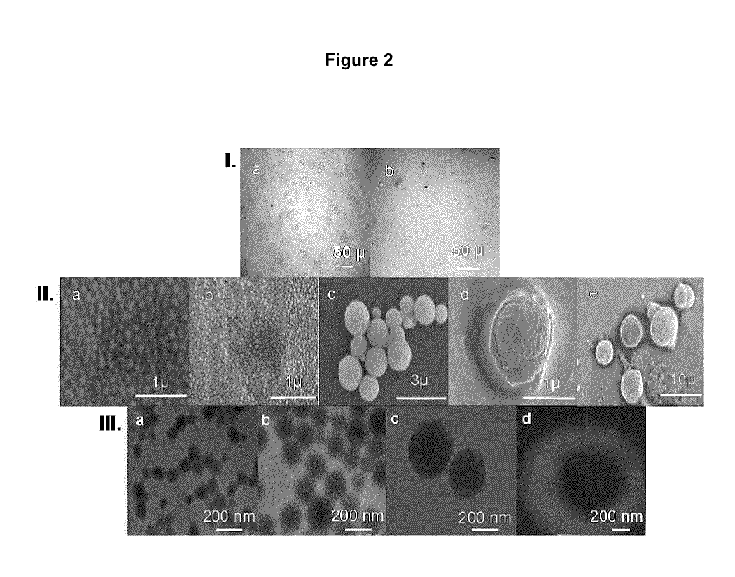

The droplet generator works on the principle of aerodynamic force where the sodium alginate solution while passing through the nozzle (diameter=0.35 mm) breaks up into micron size particles as shown in FIG. 1. A process as developed by Jayant et al., was performed to obtain uniform sized alginate microspheres (Jayant and Srivastava (2007). Dexamethasone release from uniform sized nanoengineered alginate microspheres Journal of Biomedical Nanotechnology, 3 (3), 245-53). Several instrumental parameters like nozzle size (0.35 mm), pressure (70-75 mBar), flow rate (10-18 ml/hr), distance from nozzle to cross linking solution (CaCl₂) (5-10 cm) were fixed after definition using alginate solution (2% w/v) sprayed in CaCl₂ (250 mM). The study was performed according to a modified method acquired from Jayant and Srivastava, supra. The average size of alginate microspheres using these varied parameters was 60±5 μm as demonstrated in FIG. 2 (I and II) for the various conditions.

For nano-in-micro system, nanoparticles were mixed with alginate in two ratios (1:4 and 3:4 for gelatin nanoparticles and 1:4 and 3:4 for CaCO₃ nanoparticles) and used for microsphere preparation. Particle size was also determined by changing instrumental parameters. Detailed parameters which were used and varied have been explained in Table 2.

TABLE 2
Illustrative parameters for preparation of plain alginate
microspheres and nano-in-micro particles.

| | | | | | | | | Concentration | Size of | |
|---|---|---|---|---|---|---|---|---|---|---|
| C_{alginate} | C_{CaC12} | Nozzle | Pressure | | Flow rate | | Distance | nanoparticles: | nano-in-micro | |
| (% w/v) | (% w/v) | size (μm) | (mbar) | | (ml/hr) | | (cm) | alginate | particles (μm) | |
| 1.5 | 200 | 0.35 | 150 | (5) | 130 | (2) | 5 | — | 130 | (±10μ) |
| 1.5 | 200 | 0.35 | 130 | (5) | 110 | (2) | 5 | — | 120 | (±10μ) |
| 1.5 | 200 | 0.35 | 110 | (5) | 90 | (2) | 5 | — | 100 | (±10μ) |
| 2.0 | 250 | 0.35 | 90 | (5) | 70 | (2) | 5 | — | 80 | (±10μ) |
| 2.0 | 250 | 0.35 | 55 | (5) | 30 | (2) | 5 | — | 60 | (±10μ) |
| 2.0 | 250 | 0.35 | 70 | (5) | 20 | (2) | 10 | — | 60 | (±10μ) |
| 2.0 | 250 | 0.35 | 60 | (5) | 10 | (2) | 10 | 1:4 | 60 | (±10μ) |
| 2.0 | 250 | 0.35 | 120 | (5) | 10 | (2) | 10 | 1:4 | 25 | (±5μ) |
| 2.0 | 250 | 0.35 | 300 | (5) | 15 | (2) | 10 | 3:4 | 15 | (±5μ) |
| 2.0 | 250 | 0.35 | 500 | (5) | 15 | (2) | 10 | 3:4 | 8 | (±5μ) |

Values in parenthesis represent the standard deviation for triplicate measurements.

During the present studies, the results as obtained by Jayant and Srivastava, supra. were validated for preparation of alginate microspheres and these were used for preparation of nano-in-micro particles. In general, during the present studies it was observed that increase in pressure lead to decrease in size, however sphericity was lost to some extent which could be counter balanced by increase in flow rate. In addition to this when nanoparticles were mixed in alginate solution, smaller particle sizes could be formed, which is believed to be mainly due to production of shear during atomization. Further, they also served to provide nuclei for formation of droplets which in turn lead to formation of smaller particles.

### Particle Size Analysis

Microscopic images of alginate microspheres were found to be in the size range of 30-130 μm (Table 2) while nano-in-micro alginate microspheres were found to be spherical with size ranging from 5-60 μm depending upon the parameters varied for spraying the suspension through an apparatus described in FIG. 1.

The SEM images of uncoated, coated and nano-in-micro particles of gelatin indicate that the particles formed are spherical in nature having diameter of 200 nm, 500 nm and 5-60 μm, respectively. On the other hand uncoated, coated and nano-in-micro particles of CaCO₃ indicate that the size of particles ranged from 700 nm to 1 μm, 2 μm, and 5-60 μm, respectively. The SEM results indicate a lower particle size than Dynamic light scattering (DLS) due to drying effects during sample preparation (FIG. 2(II)). TEM images reveal perfectly spherical dispersed particles. The sizes as determined from TEM images suggest the particles to be between 180 to 220 nm for plain gelatin nanoparticles while PSS/PEI coated particles are around 500 nm. CaCO₃ nanoparticles were visible as dark, solid, mesoporous with channels and pores in the structure. LBL coating was not clearly visible in case of gelatin nanoparticles, because of their small size, but in case of CaCO₃, LBL coating (2 BL) can be seen around the CaCO₃ nanoparticles (FIG. 2(III)).

Particle size and size distribution of nanoparticles and LBL coated nanoparticles were determined by dynamic light scattering using Brookhaven's instruments for gelatin nanoparticles and Nicomp® particle sizing systems for CaCO₃ nanoparticles. Both the techniques are based on a deconvolution (contin) algorithm for dynamic light scattering. Several distribution analysis methods such as intensity weighted, volume weighted and multimodal distribution analysis were applied. The results have been summarized in Table 3. A higher value of polydispersity indicates the aggregation of CaCO₃ nanoparticles due to a lower value of zeta potential (−7 mV).

TABLE 3
Particle size distribution analysis of uncoated,
coated nanoparticles and nano-in-microparticles

| Sample (Gelatin-in- | | Sample (CaCO₃-in | |
|---|---|---|---|
| alginate microspheres) | Size | alginate microspheres) | Size |
| Gelatin nanoparticles | 175 nm | CaCO₃ nanoparticles | 816 nm |
| (Uncoated) | (0.07) | (Uncoated) | (0.49) |
| Dexamethasone loaded | 201 nm | — | — |
| gelatin nanoparticles | (0.09) | | |
| [PSS/PEI]₂ coated | 581 nm | [PSS/PAH]₂ coated | 2 μm |
| gelatin nanoparticles | (0.12) | CaCO₃ nanoparticles | (±1μ) * |
| Gelatin nanoparticles | 5-60μ | CaCO₃ nanoparticles | 5-60μ |
| in alginate micro- | (±10μ) * | in alginate micro- | (±10μ) * |
| spheres | | spheres | |

Values in parenthesis in Table 3 represent the polydispersity index and the values designated with the “*” symbol with values in parenthesis represent the standard deviation.

A slight increase of ˜25 nm in the mean diameter of gelatin nanoparticles was observed in comparison to uncoated unloaded gelatin nanoparticles. Further, on addition of coating of [PSS/PEI]₂ the mean diameter increased to ˜580 nm using similar parameters. CaCO₃ nanoparticles showed a mean diameter as determined by intensity distribution of particles was found to be 816 nm (0.49).

### Zeta Potential Measurement

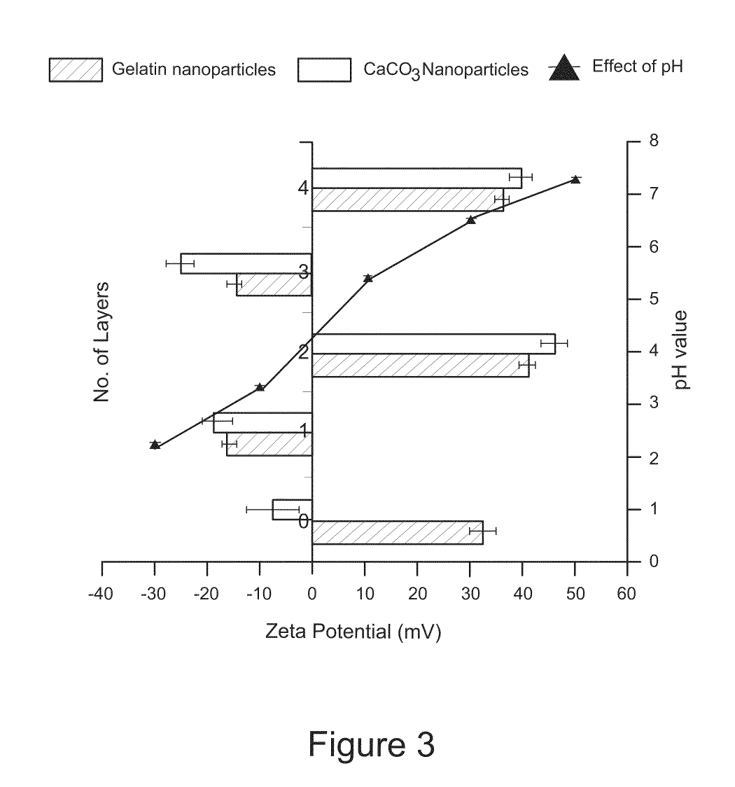

Surface charge or zeta potential in case gelatin nanoparticles was largely dependent on the pH of the suspension. A study employing different pH ranges for preparation of nanoparticles suggested that at extremes of pH gelatin nanoparticles showed finite values (negative and positive) for zeta potential, however when iso-electric point was approached the zeta potential values were reduced as shown in FIG. 3.

The effect of pH on the zeta potential of cross linked gelatin nanoparticles was studied which suggested that as the pH increased the zeta potential values decreased approaching zero till the isoelectric potential. Zeta potential was found to be between +33 to −36 mV when pH was changed from 2.2 to 8.5. This can be explained by the presence of ionisable amine groups NH⁴⁺ and carboxyl groups COO⁻. At lower pH the amine groups are protonated which gives rise to a positive charge and at higher pH the carboxyl groups are deprotonated giving a negative charge to the particles. The isoelectric point is observed to be around pH 5.5 and the particle shows high surface potential around physiological pH making it a suitable matrix for drug administration. The surface charge of gelatin nanoparticles was found to decrease with dexamethasone loading to ˜25 mV. This is evident because of the fact that phosphate salt of dexamethasone has been used which electrostatically neutralizes the positive charged amine groups. During the LBL assembly the zeta potential shifted from negative to positive on addition of PEI and vice versa on addition of PSS (FIG. 3).

In the case of CaCO₃ nanoparticles, preparation was performed in neutral pH conditions, CaCO₃ showed a slight negative charge leading to aggregation effects. In order to reduce these effects PSS was used so that the zeta potential could be increased on negative side. This formed the basis for the build of subsequent nanofilms. Alternating negative and positive zeta potentials in gelatin and CaCO₃ nanoparticles confirmed the LBL assembly over nanoparticles.

### Loading of Actives and Encapsulation Efficiency

Drug loading of nanoparticles is defined as amount of drug per mass of polymer (mg of drugs per mg of polymer), whereas encapsulation efficiency refers to ratio of amount of drug encapsulated to the theoretical loading amount used. Drug loading was determined indirectly by determining the concentration of dexamethasone which is free in the supernatant. The drug present in supernatant was determined using a standard curve prepared by UV spectroscopy at 242 nm. The regression equation y=4.698x−0.047 was plotted in range 0.02-0.5 mg/ml. Drug loading in gelatin nanoparticles was found to be 1.38, 1.26, 1.22, and 0.85% at 0.5, 0.4, 0.3 and 0.2 mg/ml concentrations, respectively. At 0.2 mg/ml the encapsulation efficiency was found to be very high (89.6%) while at 0.5 mg/ml efficiency decreased to about 58.6%. When the drug concentration increased from 0.2 to 0.3 mg/ml the efficacy decreased. Drug loading was found to be nearly constant till 0.3 mg/ml, however the efficiency is found to decrease drastically with further increase in concentration because of saturation. Similarly FITC-dex encapsulation in CaCO₃ indicated a high loading (64%) as shown by Joshi and Srivastava, 2009 supra. The entrapment/encapsulation were found to be molecular weight dependent and increase in molecular weight reduced the entrapment efficiency. Further, use of LBL coatings have shown to reduce the leaching of FITC-dex to an extent of 51% after two bilayer coatings (Joshi and Srivastava supra.).

### Confocal Laser Scanning Microscopy (CLSM)

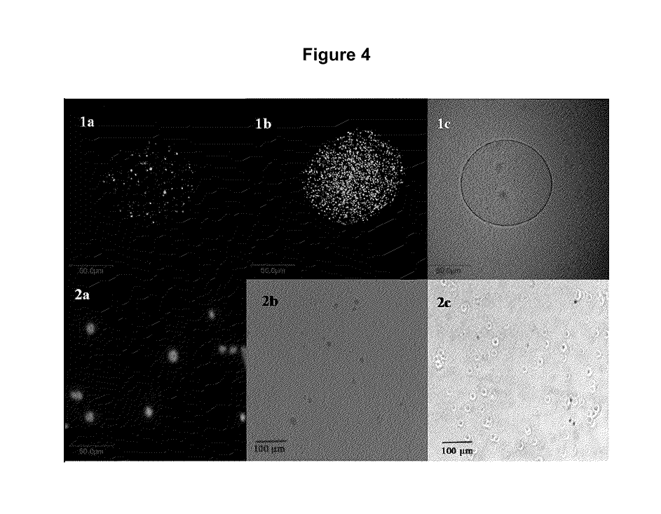

The fluorescent images of nano-in-micro system shown in FIG. 4 indicate FITC-dex loaded nanoparticles (appears as green in color in the images). The punctuate marks of fluorescence indicate non aggregated nanoparticles encapsulated in microspheres when compared against the DIC image. The fluorescent images and their corresponding DIC images show that the nanoparticles lie within the matrix. The sizes of the microspheres are more or less consistent regardless of the nanoparticles encapsulated within them. The particles appear spherical and well defined. The presence of the nanoparticles have not proven to be abrasive or structurally deforming. FIG. 4 depicts microparticle with loading of nanoparticles to see if any structural abnormality arises due to encapsulation of increasing amounts of nanoparticles. In case of CaCO₃ loaded with FITC dye, it was found that it was not retained in CaCO₃ nanoparticles due to mesoporous nature of CaCO₃. However when CaCO₃ was loaded with FITC-dex (70 kDa) it was found to be retained to a greater extent due to its high molecular weight and the adsorption of the macromolecule on the CaCO₃ nanoparticles.

### Fourier Transform Infra-Red Spectroscopy (FTIR)

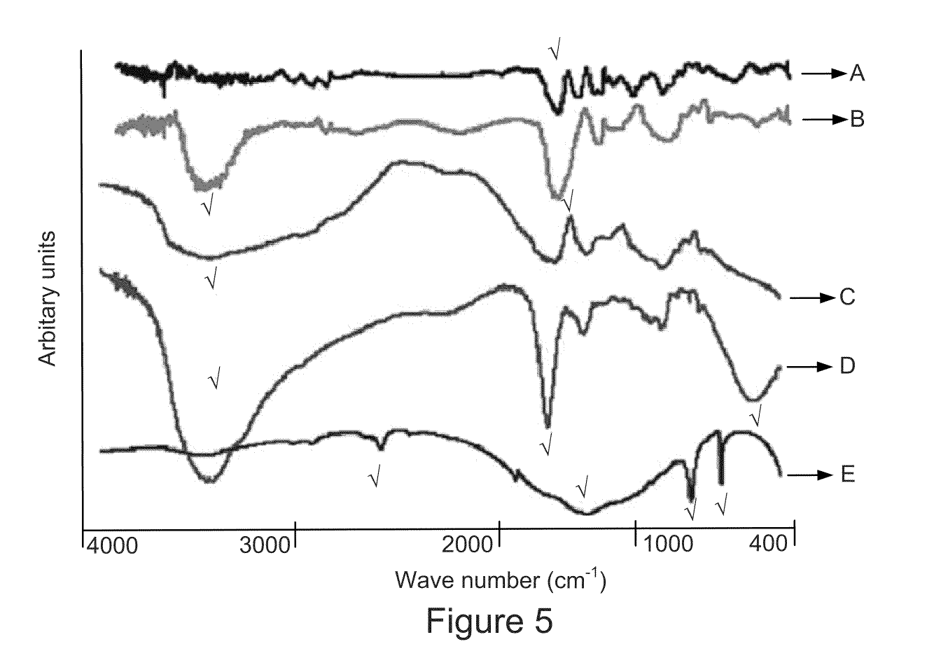

Gelatin being a proteinaceous molecule C═O and NH bond stretching vibrations act as an index of its presence. The peak at 3450 cm⁻¹ which is indicative of N—H bonds present in gelatin was observed in both gelatin and gelatin-in-alginate hybrid microspheres. The peaks at 1400 cm⁻¹ indicate CO bond stretching and as both gelatin and alginate contains these groups similar peaks are observed in the two graphs (FIG. 5). The major characteristic peak of CO stretching at 1400 cm⁻¹ is due to the concentration of CO bond both in gelatin and alginate leading to a sharp, high intensity peak. The characteristic peaks of gelatin and alginate also correspond to the research carried out by Bajpai et al. and Wang et al. (Bajpai and Choubey (2006). Design of gelatin nanoparticles as swelling controlled delivery system for chloroquine phosphate. Journal of Material Science and Material Medicine, 17, 345-358., Wang et al. (2008). Synthesis and characterization of CdTe quantum dots embedded gelatin nanoparticles via a two-step desolvation method. Material Letters, 62, 3382-84).

On the other hand, CaCO₃ nanoparticles show peaks at 3442 cm⁻¹, 2925 cm⁻¹, 1490 cm⁻¹, 1437 cm⁻¹, 877 cm⁻¹ (Goma and Hund (2008). Amorphous calcium carbonate in form of spherical nanosized particles and its application as fillers for polymers. Materials Science and Engineering, 477 (1-2), 217-225; Ma and Zhou (2008). Study on CaCO₃/PMMA nanocomposite microspheres by soapless emulsion polymerization. Colloids and Surfaces A: Physicochemical and Engineering Aspects, 312 (2-3), 190-194). In case of CaCO₃ in alginate hybrid microspheres, two peaks (1437 and 877 cm⁻¹) characteristic of CaCO₃ nanoparticles were observed indicating the presence of CaCO₃ in the hybrid microspheres (FIG. 5). The peak at 1437 cm⁻¹ becomes more prominent and sharp due to the synergistic effect of C═O of CaCO₃ and alginate. Due to a higher concentration of alginate in CaCO₃ in alginate particles the OH stretching peak at 3400 cm⁻¹ appears broadened. FTIR studies indicate that in both systems containing gelatin in alginate and CaCO₃ in alginate no new peaks corresponding to any chemical bonding arise but the presence of characteristic peaks of components shows that both the components are physically present in the matrix. The reduced intensities of peaks corresponding to CaCO₃ and gelatin in hybrid nano-in-micro system are due to lower concentration ratios in the microspheres.

### Drug Release Studies

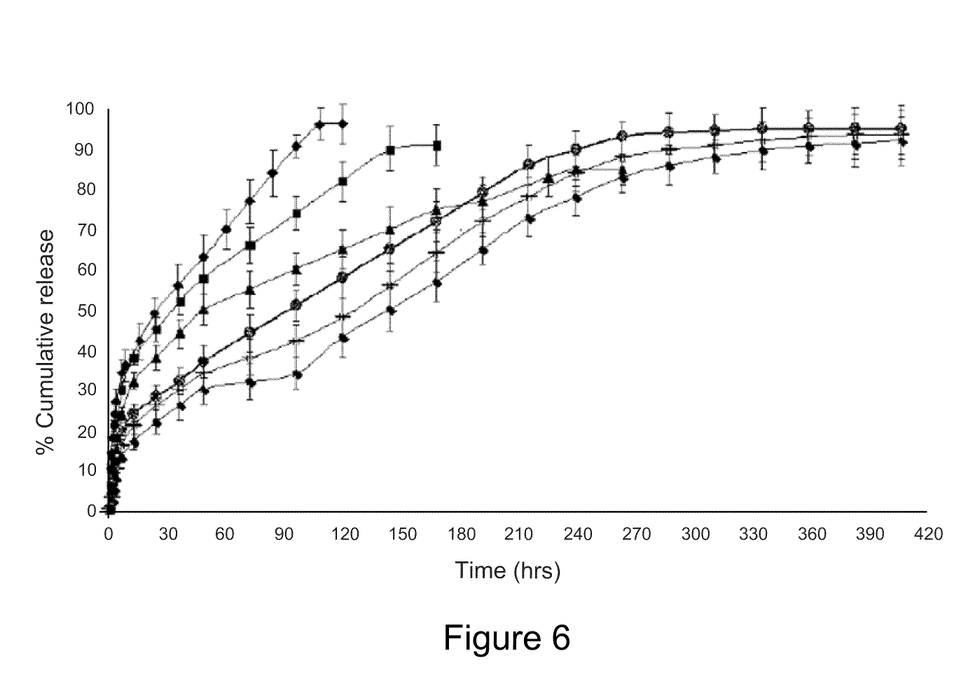

In release studies, release profiles of uncoated nanoparticles, LBL coated nanoparticles (1 bilayer (1 BL) and 2 bilayer (2 BL)), nano-in-micro hybrid particles containing (1:4 and 3:4 loading of nanaoparticles: alginate) were compared as shown in FIG. 6. Gelatin nanoparticles showed zero order release behavior for dexamethasone after an initial burst release period which lasted for 3 hrs. During the burst release period up to 25% of the drug was released. Following the burst release a steady zero order release kinetics up to 4 days was observed with a correlation coefficient of 0.994. The biphasic pattern of drug release is characteristic of matrix diffusion kinetics, as is expected in a nanoparticle based drug delivery system. 97% of the initial drug loading was found to be released in duration of 96 Hours. LBL coated (2 BL) nanoparticles showed reduced burst release from the nanoparticles. This could be attributed to a decrease in surface associated drug in LBL coated particles. 2 BL coated particles released only 15% of the drug in contrast to 18% of the 1 BL coated particles in first 3 hours. The sustained release of drug after the burst release lasted for 6 days in the 1BL coated particles, releasing 90% of the initially loaded drug. However in case of 2 BL coated particles the release could be sustained up to 9 days showing 85% of initial loading. The release of dexamethasone could be prolonged to an extent of 50% and 100% in 1 BL and 2 BL coated nanoparticles, respectively. Although not intending to be limited by any theory, this increase in the release period is believed to be due to the presence of an additional diffusion layer which acts as a barrier.

The comparison of release profiles of nano-in-micro systems (1:4 and 3:4 ratios) against plain alginate microsphers shows that initial burst release was 21% and 17% in case of 1:4 and 3:4 configurations, respectively. The initial burst release was clearly reduced when compared against plain alginate microsphere wherein 24% of dexamethasone was released during burst release period. This is attributed to lack of surface adhered drug alginate microspheres containing nanoparticles. The immediate reservoir of drug is in the open pores of the alginate matrix which can be diffuse out instantaneously. Lower percentage of burst release is indicated due to this phenomenon in both the nano-in-micro configurations. The sustained release phase of the three configurations (plain alginate microspheres, 1:4 and 3:4 nano-in-micro systems) showed significant differences in the release pattern.

The plain drug loaded alginate particles show a normal zero order release profile, as expected; the nano-in-micro systems showed a varied release profile. Both of the nano-in-micro systems showed a decrease in % cumulative release during 96-100 hrs, after which it follows a regular zero order kinetics. This variation from the normal zero order profile can be explained by the presence of two different matrices in a single system which individually exhibit different release profiles. In both of the nano-in-micro configurations, the drug released from nanoparticles is not held by the alginate matrix, but instead the drug appears to diffuse through the pores of the alginate matrix.

As it was observed that gelatin nanoparticles released its contents in a 4 day period, after 4 days, the alginate pores in the matrix hold the drug in the form of a reservoir after gelatin is degraded. The nano-in-micro system prolongs the release up to 14 days and 16 days in the case of 1:4 and 3:4 systems, respectively when compared against drug loaded alginate microspheres which maintained the release for 11 days. This showed superiority of the nano-in-micro system over other nanoparticle and microparticle matrix based drug delivery systems in terms of reduced burst release and prolonged release.

Dexamethasone release from gelatin nanoparticles and nano-in-micro systems indicated a decrease in burst release in the order: uncoated (24%), >coated (18%), >nano-in-micro system (1:4 ratio) (17%), respectively. The sustained release decreased in order of nano-in-micro (1:4 ratio) (14 days)>coated nanoparticles (9 days)>uncoated nanoparticles (4 days) for 95% drug release. FITC-dex loading and release from CaCO₃ nanoparticles was found to be molecular weight dependent.

The method of nano in micro encapsulation based on air driven atomization can be used for encapsulation of nanoparticles in alginate based microspheres. Microsphere production was achieved by cross linking atomized droplets of alginate containing nanoparticles. Various sizes ranging from 5-60 μm can be achieved by alteration of instrumental and sample parameters. Encapsulation of drug loaded nanoparticles has been performed to achieve a release which can be better controlled in comparison to uncoated and LBL coated nanoparticles. On the other hand, CaCO₃ nanoparticles being meso-porous in nature can be loaded with macromolecules like enzymes which can serve as an efficient matrix for preparation of biosensors. These findings suggest that the nanoparticles inside alginate matrix are superior candidates for encapsulation of drug/macromolecules owing to the biocompatible nature of alginate.

(c) Confocal Laser Scanning Microscopy (CLSM). Fluorescent images of FITC labeled dextran of different molecular weights viz. 70 KDa, 150 KDa and 500 KDa were examined using confocal microscopy. Confocal micrographs of microparticles were taken with a flow view confocal laser scanning microscope (CLSM) equipped with a krypton-argon laser (Olympus FluoView™, Japan). An inverted microscope was used which was equipped with an oil immersion objective lens (60×). The standard filter settings for fluorescence excitation (488 nm) and emission were used.

(d) X-ray Diffraction (XRD). XRD studies were conducted at ambient conditions using Philips X′Pert Diffractometer (model PW 3040/60) equipped with a 2θ compensating slit, using Cu Kα radiation (λ=1.5406 Å) at 40 kV, 30 mA passing through a Ni filter. The instrument was calibrated for accuracy of peak positions using a silicon pellet. Powder samples (˜100 mg) were placed onto sample holder and hand-leveled with a clean glass slide. Data was collected in a continuous scan mode with a step size of 0.0170° and step time of 15.18 s over an angular range of 3° to 40° 2° θ. The data obtained was analyzed using diffraction software. Bare CaCO₃, PSS doped CaCO₃ at two different concentrations were dried and analyzed to obtain XRD spectrum.

(e) Fourier Transform Infra-Red Spectroscopy (FT-IR). Samples including CaCO₃, PSS, CaCO₃-PSS doped particles, were dried to obtain their dry form. Samples were thoroughly ground with dried KBr and discs were prepared by compression. FTIR analysis of the samples was performed using FTIR spectrometer (Nicolet Instruments Corporation, Magna 550, USA). Spectra were obtained on the spectrometer from 400 to 4000 cm⁻¹.

### Characterization

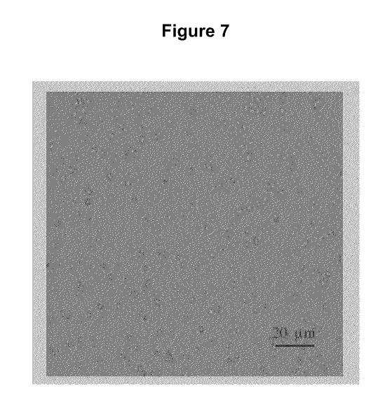

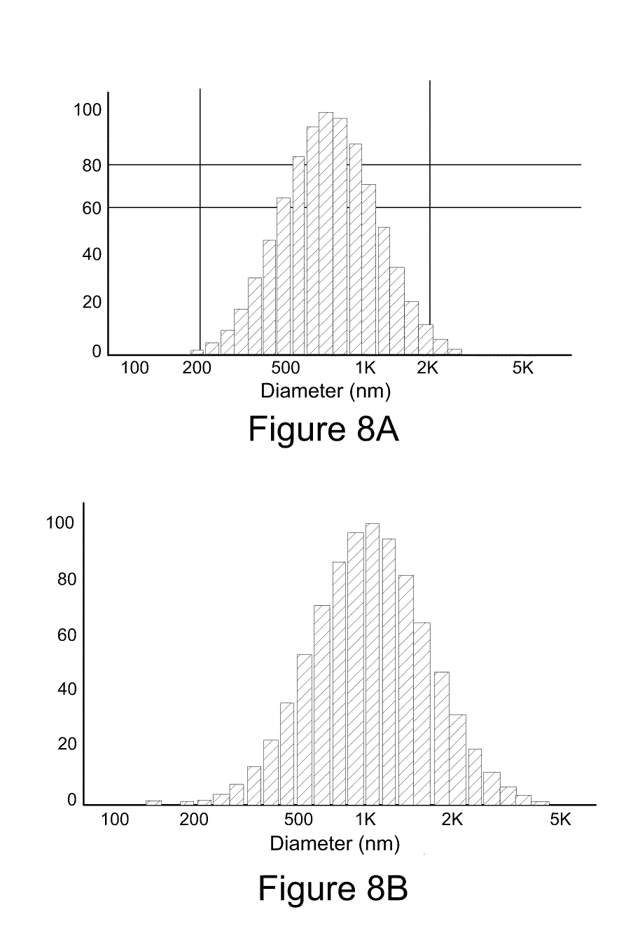

(a) Particle Size Analysis. Optical microscopic images of microparticles (FIG. 7) showed the presence of spherical particles of uniform size having diameter of 2 μm as determined by the particle sizing software. The CaCO₃ nanoparticles precipitated in the absence of PSS showed greater aggregation potential with particle size ranging from 2-7 μm. PSS exhibits micellization effect which causes formation of smaller nanoparticles (Yue et al. Microporous Mesoporous Mater. 113, 1 (2008)) in comparison to precipitation in distilled water (in absence of PSS). The presence of PSS during precipitation also leads to uniform spherical shape of the nanoparticles. The size distribution determined using particle sizing software indicated particle size of ˜1 μm. Further, Gaussian size distribution was obtained using Nicomp® particle sizing systems (FIG. 8). It uses a proprietary high resolution deconvolution algorithm working on a similar principle of dynamic light scattering. Several distribution analysis methods such as intensity weighted, volume weighted and multimodal distribution analysis were applied. The results of intensity and volume weighted Gaussian distribution analysis showed that the mean diameter of the particles lies in the range of 816 nm (Variance: 0.49) and 1037.5 nm (Variance: 0.49), respectively.

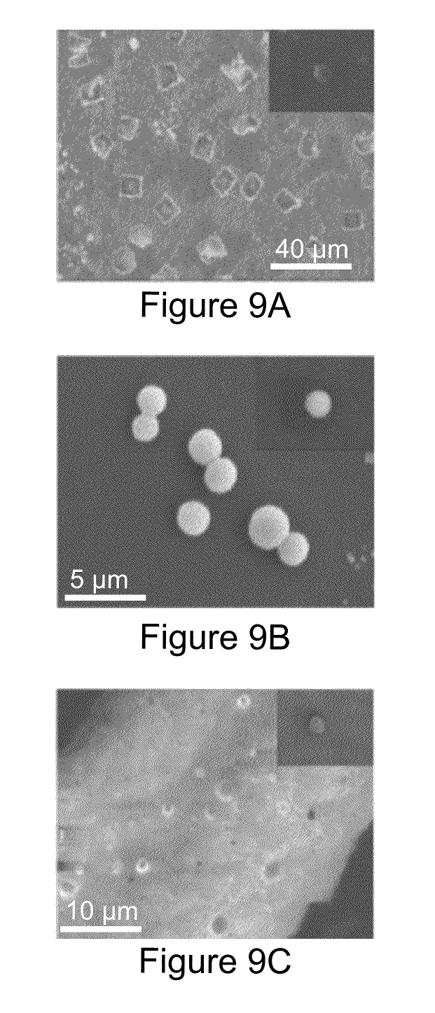

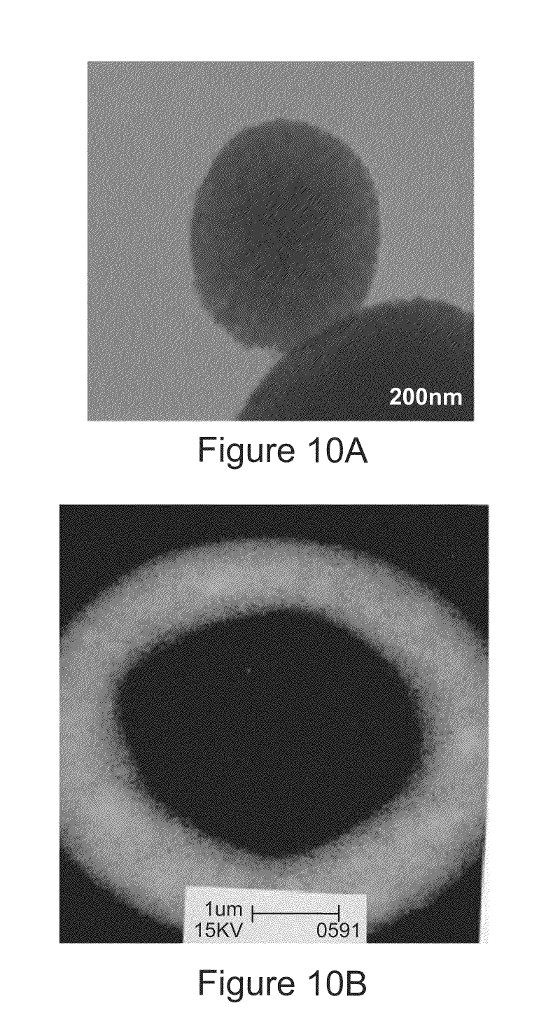

SEM images of CaCO₃ nanoparticles without PSS and PSS doped nanoparticles are presented in FIG. 9(a). The Bare CaCO₃ (without PSS) nanoparticles showed different crystal morphologies with size ranging from 2 μm to 7 μm. Also the crystal arrangement formed is of calcite nature which appears like a cube or hexagon in shape. This form is the most stable form to which all meta-stable forms of CaCO₃ nanoparticles like vaterite, aragonite and amorphous CaCO₃ convert after certain period of time (Brecevic and Kralj, Croatica Chemica Acta 80, 3 (2007)). On the other hand, the PSS doped CaCO₃ nanoparticles show the presence of spherical crystal arrangement (FIG. 9(b)). Due to the presence of PSS in the meso-porous structure of the spherical crystals, aggregation potential of the crystals is reduced. The presence of polyelectrolyte leads to formation of spherical particles as also reported by Kawaguchi et al. Thus a considerable stabilization of nanoparticles to recrystallization was observed with PSS. LBL assembly on the nanoparticles shows a typically rough surface. A clear difference was visible in comparison with the uncoated nanoparticles (FIG. 9(c)) (Kawaguchi, supra). TEM image of uncoated CaCO₃ nanoparticles exhibited mesoporous nature having channel like structures (FIG. 10(a)). The TEM image for LBL coated CaCO₃ nanoparticles showed the presence of a hard core surrounded with less dense coating, which probably signifies the presence of polyelectrolyte coatings over the nanoparticles. The particle size of the nanoparticles was also confirmed from the TEM image having size in the range 1-2 μm and the coating i.e., the PSS doping and the LBL self-assembly constitute thickness of about 1 μm (FIG. 10(b)).

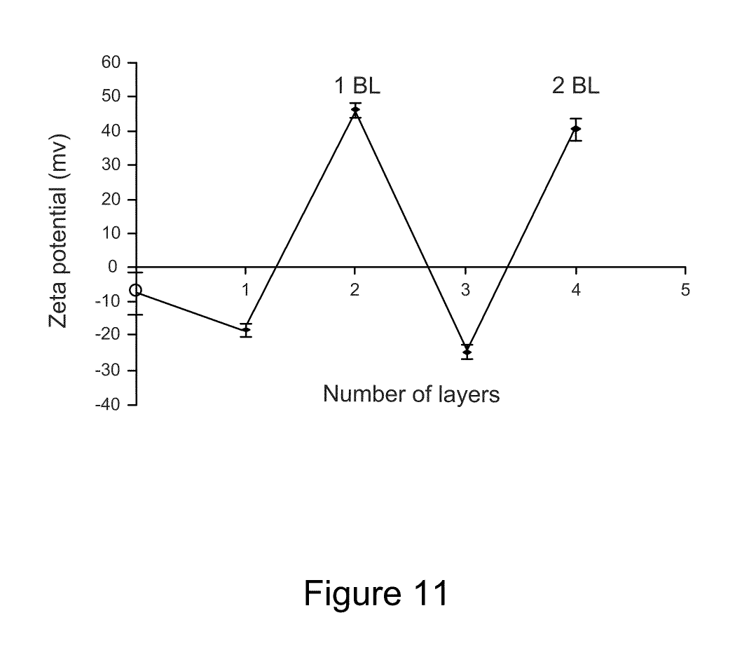

(b) Zeta Potential Measurement. Zeta potential measurements of uncoated and coated nanoparticles were performed in order to confirm the LBL assembly over the nanoparticles. Zeta potential of bare CaCO₃ nanoparticles was found to be −7 mV. PSS doped nanoparticles showed a zeta potential value of −18.48 mV, which showed increased stability in suspension form in comparison to the bare CaCO₃ micro/nanoparticle suspension. PSS was selected because of its surface active properties in spite of the slight negative charge on the particles (Antipov et al. Colloids and Surfaces A: Physicochem. Eng. Aspects 224 (2003)). The presence of PSS within the meso-porous structure serves as a template having necessary anionic charge for the deposition of positively charged polyelectrolyte PAH. Subsequent attachment of different layers showed corresponding changes in the zeta potential as shown in FIG. 11. In order to determine the effect of concentration of PSS on the zeta potential and consequently on the stability, different concentration of PSS were incubated during precipitation. Zeta potential for 0.125, 0.25 and 0.5% PSS doped CaCO₃ nanoparticles was found to be −21.9 (0.02) mV, −23.7 (0.32) mV and −21.5 (0.54) mV, respectively. This indicates that there is no significant change in zeta potential values with increasing concentrations in the formation of PSS doped nanoparticles. Subsequently, 0.25% PSS was selected in preparation of PSS doped nanoparticles.

### Macromolecular Encapsulation

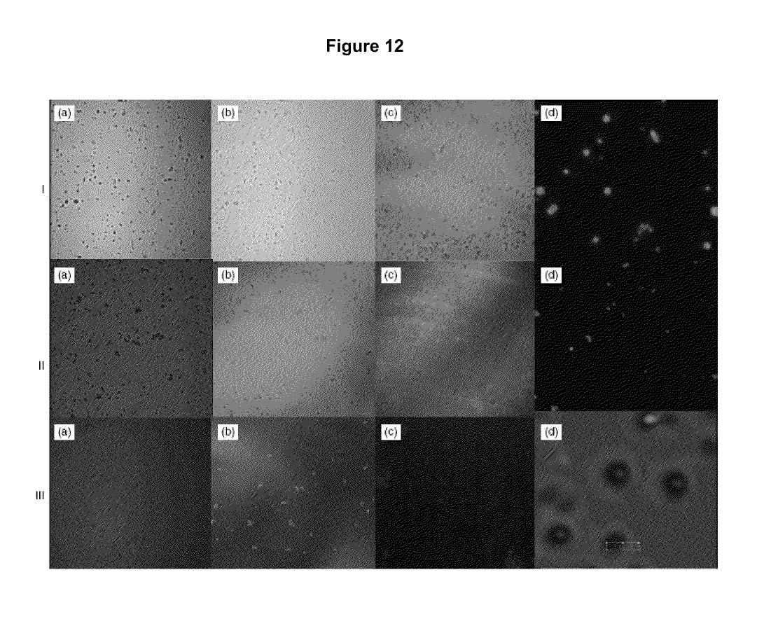

Macromolecules have been loaded on CaCO₃ nanoparticles conventionally, either during precipitation, on preformed nanoparticles, or on preformed polyelectrolyte coated nanoparticles. Encapsulation of FITC-dextran on preformed CaCO₃ nanoparticles was confirmed using fluorescent microscopic imaging (FIG. 12). The fluorescence images demonstrate the presence of FITC-dextran within the micro/nanoparticle matrix. Fluorescent images of CaCO₃ nanoparticles also show different molecular weight of FITC-dextran encapsulated during preparation and washing of nanoparticles. The amount of FITC-dextran encapsulated was found to be dependent on the molecular weight of FITC-dextran and the porosity of the particles. In case of 500 KDa FITC-dextran, encapsulation was found to be much lower in comparison to 70 KDa and 150 KDa FITC-dextran (FIG. 12 (IIId)). Fluorescence spectro-photometric study for CaCO₃ nanoparticles also confirmed encapsulation of FITC-dextran indicated by decrease in fluorescence intensity in supernatant solution in comparison to blank standard solution of FITC-dextran. In order to establish the instrument reproducibility multiple readings of fluorescence emission spectra of a single sample of enzyme solution was captured at a single wavelength utilizing the intrinsic fluorescence property of enzyme solutions due to the tyrosine and tryptophan residues of the protein. The fluorescence emission intensity for the sample was found to be 67.3 (0.79) for n=6 measurements. Calibration curves were prepared using different molecular weight of FITC-dextran, and thereby used for quantification of encapsulation of FITC-dextran on CaCO₃ nanoparticles (Table 4).

TABLE 4
Calibration curves of different molecular weights of FITC-dextran

| S. No. | FITC-dextran molecular weight (KDa) | Range (mg/ml) | R² |
|---|---|---|---|
| 1 | 70 | 0.02-0.12 | 0.977 |
| 2 | 150 | 0.01-0.14 | 0.992 |
| 3 | 500 | 0.04-0.2 | 0.977 |

The encapsulation efficiency of 70 KDa FITC-dextran occurs to an extent of (n=3) 68.6% (5.41), 150 KDa FITC-dextran occurs to an extent of (n=3) 33.1% (1.21) and 500 KDa occurs to an extent of 3.9 (0.3) % upon loading with 0.2 mg/ml solution of FITC-dextran. Encapsulation efficiency of macromolecules on preformed CaCO₃ nanoparticles is a function of both adsorption and entrapment.

Macromolecular Release from the CaCO₃ Nanoparticles

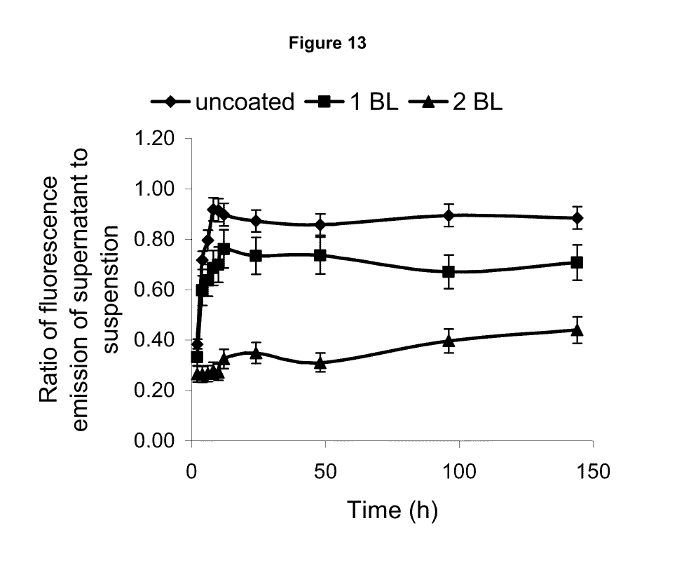

The release of FITC-dextran (70 KDa) was found to be higher from the uncoated CaCO₃ nanoparticles in comparison to leaching from one and two bilayer coated nanoparticles. The release in the case of uncoated particles shows highest rate in the eight days of the experiment. However a burst release of FITC-dextran occurs in the first two days, which occurs due to an adsorbed layer of FITC-dextran on to the nanoparticles. This phenomenon was reduced in case of coated nanoparticles owing to the absence of the adsorbed layer. Therefore ratio of fluorescence emission of supernatant to suspension decreases with increase in number of coatings (FIG. 13). The release profile generated in the case of uncoated nanoparticles is a function of adsorbed and encapsulated fraction of FITC-dextran. However, in the case of 1 BL and 2 BL the release profile majorly constituted the encapsulated fraction of FITC-dextran. LBL coating causes a decrease in the net fluorescence emission intensity of the one bilayer coated and the two bilayer coated particles in comparison to the uncoated nanoparticles. The decrease in release due to LBL coatings was estimated by determining and comparing the slopes of release up to 2 days. The values of slopes obtained from the linear regression analysis of up to 2 days were found to be 0.0631, 0.041 and 0.0014 for uncoated, One bilayer (1 BL) and Two bilayer (2 BL) nanoparticles, respectively. This shows that although the encapsulated amount is less for one bilayer coated nanoparticles and two bilayer coated nanoparticles, LBL assembly has successfully decreased the leaching of the high molecular weight compound in the order: uncoated nanoparticles>one bilayer coated>two bilayer coated nanoparticles.

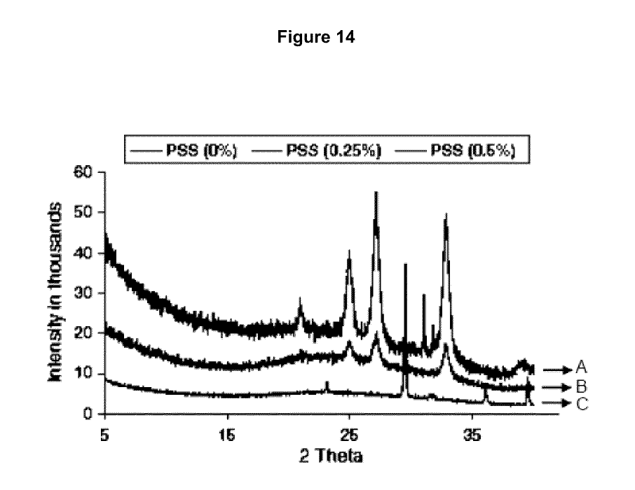

(c) XRD. Powder XRD profiles clearly indicate that in presence of polyelectrolyte, the crystal arrangement into which CaCO₃ nanoparticles precipitate is altered from a calcite conformation to a spherical vaterite structure. This fact is also substantiated by Kawaguchi et al.: spherical crystals can be formed in the presence of polymers like polystyrene. Different PSS concentrations were used to study the crystal arrangement obtained. Calcite structure has characteristic peaks at 2 theta values of 3.06, 23.16, 29.51, 36.07, and 39.52, where as vaterite structure has characteristic peaks at 2 theta values of 20.99, 24.95, 27.14, 31.04, 31.78, and 32.82 (Kawaguchi, supra). FIG. 14 clearly indicates that in presence of PSS only one type of crystal arrangement is formed which is proved by the absence of peaks corresponding to the calcite form. Further, as the concentration of PSS is increased the concentration of the spherical polymorph formation is increased.

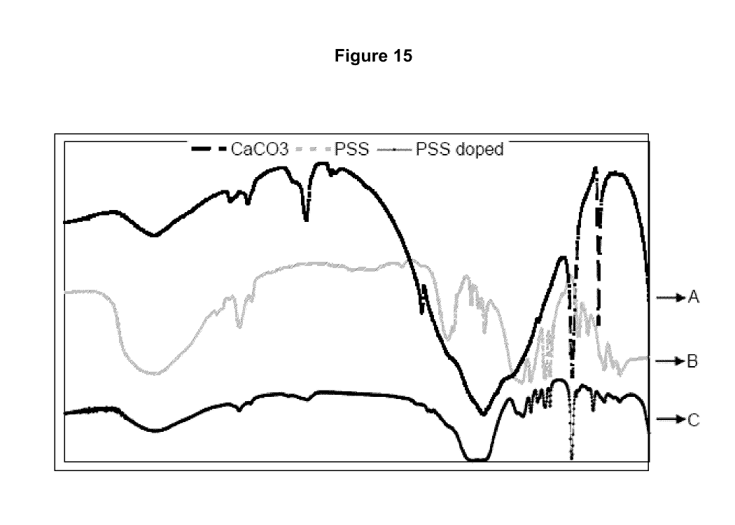

(d) FTIR. FTIR spectra of CaCO₃ nanoparticles showed characteristic peaks at 1417 cm⁻¹ and 877 cm⁻¹ where as for PSS it shows at 3448 cm⁻¹, 2923 cm⁻¹, 1601 cm⁻¹ and 1496 cm⁻¹ (FIG. 15). In case of PSS doped CaCO₃ nanoparticles, although no new peaks corresponding to any chemical bonding arise but the presence of characteristic peaks of both CaCO₃ and PSS shows that both the components are physically present in the matrix. PSS doped CaCO₃ particles show peaks at 3442 cm⁻¹, 2925 cm⁻¹, 1490 cm⁻¹, 1437 cm⁻¹, 877 cm⁻¹ (Ma et al. Colloids and Surfaces A: Physicochemical and Engineering Aspects 312, 2 (2008)). All the peaks are present either in CaCO₃ or PSS, indicating presence of both the components. The reduced intensities of peaks corresponding to pure PSS may be due to lower concentration in the final PSS doped particles in comparison to high concentration of CaCO₃.

Meso-porous nature of the crystals, support high loading efficiency of high molecular weight compounds. Since the encapsulation is dependent on molecular weight of the encapsulant molecules, different molecular weights of high molecular weight compounds of FITC-dextran (70, 150, and 500 KDa). Further, LBL nanoengineering technique can be used to reduce the leaching of macromolecules from nanoparticles demonstrating the potential of loading macromolecules in the nanoparticles for development of stable enzyme based biosensors.

The nanoparticles being meso-porous in nature can be loaded with high molecular weight macromolecules. Qualitative and quantitative estimation of encapsulation for FITC-dextran (70 KDa, 150 KDa and 500 KDa), loading on the nanoparticles was analyzed using fluorescence spectroscopy. FITC-dextran showed encapsulation of 64%, 33.2%, 3.9%, for FITC-dextran 70 KDa, 150 KDa, 500 KDa, respectively. LBL self-assembly to prevent release of encapsulants was confirmed by measuring the electrophoretic mobility. The alternating negative and positive zeta potential values confirmed the successful coating of PSS and PAH, respectively on the nanoparticles. The encapsulated FITC-dextran release from uncoated and coated nanoparticles was monitored; the results suggest that LBL coatings decreased the release in comparison to the uncoated particles. These findings suggest that the CaCO₃ nanoparticles are superior candidates for encapsulation of macromolecules owing to the micro porous nature of the nanoparticles and subsequently for development of stable biosensors.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of preparing a microsphere with one or more entrapped nanoparticles, comprising:
atomizing a suspension comprising a polysaccharide and one or more nanoparticles into a solution comprising a cross linking agent, thereby preparing a microsphere with one or more entrapped nanoparticles.

2. The method of claim 1, wherein the polysaccharide is an alginate.

3. The method of claim 2, wherein the alginate is from about 0.01 w/v concentration to about 5% w/v concentration of alginate.

4. The method of claim 1, wherein the nanoparticle comprises a material selected from the group consisting of: inorganic material, polymeric material, and combination thereof.

5. The method of claim 4, wherein the inorganic material comprises hydroxyl apatite, calcium phosphate, calcium carbonate, gold, iron, silica and magnetic material.

6. The method of claim 4, wherein the polymeric material comprises gelatin, poly(lactic-co-glycolic acid), and peroxalate.

7. The method of claim 1, wherein the nanoparticle is prepared by methods selected from the group consisting of desolvation, emulsification, atomization, precipitation, and sedimentation.

8. The method of claim 1, wherein the nanoparticle is a coated nanoparticle with multiple layers.

9. The method of claim 8, wherein the coated nanoparticle comprises layers of poly cations and poly anions.

10. The method of claim 8, wherein the multiple layer coated nanoparticle is made from layer by layer assembly of polyelectrolytes on the nanoparticle.

11. The method of claim 1, wherein the suspension and/or the nanoparticle comprises a fluorescent agent.

12. The method of claim 1, wherein the suspension and/or the nanoparticle comprises one or more therapeutic agents.

13. The method of claim 1, wherein a ratio of the nanoparticle and the polysaccharide is in a range of about 1:10 to about 10:1.

14. The method of claim 1, wherein a size of the nanoparticle ranges from about 1 nm to about 500 nm.

15. The method of claim 1, wherein a size of the microsphere is in a range of about 1 to about 130 μm.

16. The method of claim 1, wherein the cross linking agent is a divalent and/or trivalent metal salt.

17. The method of claim 1, further comprising adding a chemical reagent to the microsphere wherein the chemical reagent removes the entrapped nanoparticles and gels the microsphere internally.

18. The method of claim 1, wherein the cross linking agent is calcium chloride.

19. The method of claim 1, wherein the atomization comprises using a spray nozzle system of a droplet generator.

20. The method of claim 1, wherein the atomization comprises syringe extrusion, coaxial air flow method, mechanical disturbance method, electrostatic force method, or electrostatic bead generator method.

21. The method of claim 1, wherein the atomization comprises spraying the suspension through a nozzle of an air driven droplet generating encapsulation unit.

22. The method of claim 21, wherein a shape or a size of the microsphere is varied by varying one or more parameters selected from the group consisting of: nozzle diameter; flow rate of the spray; pressure of the spray; distance of the solution comprising the cross linking agent from the nozzle; concentration of the polysaccharide solution; and concentration of the cross linking agent.

23. A composition comprising a microsphere with one or more entrapped nanoparticles, the microsphere prepared by the method comprising:
atomizing a suspension comprising a polysaccharide and one or more nanoparticles into a solution comprising a cross linking agent, and an excipient.

24. A method for delivering one or more therapeutic drugs to a subject in need thereof, comprising:
administering to a subject a composition comprising a microsphere with one or more entrapped nanoparticles,
wherein the microsphere with one or more entrapped nanoparticles is prepared by atomizing a suspension of a polysaccharide and one or more nanoparticles into a solution comprising a cross linking agent to yield the entrapped one or more nanoparticles,
wherein the nanoparticle comprises one or more therapeutic drugs, thereby delivering the one or more therapeutic drugs to the subject.

25. A kit, comprising:
a microsphere prepared in accordance of claim 1.
